(19) United States
(12) Patent Application Publication
(10) Pub. No.: US 2021/0335496 A1
(43) Pub. Date: Oct. 28, 2021

(54) System, Method and Computer Readable Medium to Estimate the Post-Treatment Blood Cell Sub Type Count in Patients Treated via Radiation Therapy

(71) Applicant: University of Virginia Patent Foundation, Charlottesville, VA (US)

(72) Inventors: Krishni Wijesooriya, Charlottesville, VA (US); Seth Wijesooriya Liyanage, Stanford, CA (US); Jonathan Colen, Chicago, IL (US)

(73) Assignee: University of Virginia Patent Foundation, Charlottesville, VA (US)

(21) Appl. No.: 17/237,958

(22) Filed: Apr. 22, 2021

Related U.S. Application Data
(60) Provisional application No. 63/014,226, filed on Apr. 23, 2020.

(51) Int. Cl. G16H 50/50; G16H 10/60; G16H 20/40; G16H 15/00; G16H 30/20; G16H 30/40; G16H 50/70; G06T 11/00

(52) U.S. Cl. CPC G16H 50/50; G16H 10/60; G16H 20/40; G16H 15/00; A61N 5/1039; G16H 30/40; G16H 50/70; G06T 11/008; G16H 30/20

(57) ABSTRACT

A system, method, and computer readable medium for estimating the patient specific and plan specific radiation dose delivered to any type of circulating blood cell type or sub-type, such as, but not limited to, T lymphocytes, B lymphocytes, natural killer cells, erythrocytes, or neutrophils, and predicting time dependent fractional blood count and cell kill following radiation therapy treatment. Additionally, the system, method, and computer readable medium provide parameters such as a dose dependent lymphocyte kill function and average net release rate of new lymphocytes into circulating blood, which also includes the proliferation of existing cells and natural death of lymphocytes in blood. Determining lymphocyte kill following Stereotactic Body radiation therapy (SBRT) to lung tumors is an example of an application of the system, method, and computer readable medium.

### CROSS REFERENCE TO RELATED APPLICATIONS

The present application claims benefit of priority under 35 U.S.C § 119 (e) from U.S. Provisional Application Ser. No. 63/014,226, filed Apr. 23, 2020, entitled “System, Method and Computer Readable Medium to Estimate the Post-Treatment Blood Cell Sub Type Count in Patients Treated via Radiation Therapy”; the disclosure of which is hereby incorporated by reference herein in its entirety.

### FIELD OF INVENTION

The present invention relates to a computational technique for predicting, patient specific and radiation plan specific, any type of blood cell kill related to radiation therapy treatments. As an example, lymphocyte kill following Stereotactic Body radiation therapy (SBRT) to lung tumors is described here.

### BACKGROUND

Radiation Therapy (RT) is known to modulate the blood cells and immune system, contribute to the generation of anti-tumor T cells, and stimulate T cell infiltration into tumors. However, this anti-tumor activity may be offset by radiation-induced immunosuppression and lymphopenia, which may result in lower tumor control and survival. Lymphopenia caused by radiation therapy was first described in the early 20th century, just shortly after the discovery of x-rays (1). It has been demonstrated that radiation can induce lymphopenia in the absence of concomitant chemotherapy or steroids and even when neither bone marrow nor lymphatic tissue is included in the treatment field. Studies have shown that irradiation of the brain, which includes minimal bone marrow in the calvarium and no lymphatic tissue, can cause a greater than 60% decrease in lymphocyte count (2). One study demonstrated that irradiation of circulating blood with cesium placed inside a shielded dialysis unit caused a 60% to 80% drop in the number of circulating lymphocytes that persisted for many years after radiation exposure (3). Therefore, it is well established that irradiation of circulating blood reduces lymphocyte counts significantly.

Recent studies have shown a correlation between Treatment-Related Lymphopenia (TRL) and inferior survival in patients with glioblastoma, advanced stage non-small cell lung cancer (NSCLC), pancreatic cancer, and squamous cell carcinoma of the head and neck (4)(5)(6)(7)(8). FIG. 1 shows data from a study published in 2015 (9), where investigators collected and analyzed data from four independent solid tumor sites from each of 297 patients with newly diagnosed malignant glioma, resected and un-resected pancreatic cancer, and stage III NSCLC. The investigators recorded lymphocyte counts, prognostic factors, treatment and survival. They defined TRL as <500 cells/mm³ and found an increased risk for death attributed to TRL in each cohort. They observed severe TRL in 40% of patients two months after the initiation of chemoradiation and found that it was independently associated with shorter survival from tumor progression, as shown by FIG. 1.

In a study looking at patients treated for squamous cell carcinoma of the head and neck, it was shown that at two months 60% of patients had severe TRL, which was independently associated with earlier disease progression than those with lower TRL numbers (HR 5.75, p=0.045) (8). In another study that examined the effect of steroids and RT on the lymphocyte count in patients treated for primary brain tumors, it was found that 17 of the 70 (24%) patients had CD4 counts that decreased to <200/mm³ and that these patients were more likely to be hospitalized (41% vs 9%, p<0.01) with 23% vs 4%, p<0.05 hospitalized for infection (10). FIG. 2 shows data from another study, with Kaplan-Meir curves (11) showing survival for 133 patients that underwent treatment for locally advanced pancreatic cancer and were stratified by severe lymphopenia (Total Lymphocyte Count (TLC) <500 cells/mm³) two months after starting radiation therapy. They reported a statistically significant survival difference for patients with higher TLC, as shown by FIG. 2. Median survival for patients with severe lymphopenia at two months after starting RT was 12.4 months (95% CI: 8.7 -16.1) versus 15.2 months (95% CI: 12.7-17.9) for patients with TLC>500 cells/mm³ (P=0.055) (12).

In addition to their vital function in the body's general defenses against infections, lymphocyte sub types also play very important roles in tumor suppression. It has been shown that the expressions of CD3+ and CD4+ sub types of lymphocytes were significantly associated with overall survival of NSCLC patients (13). CD8+ and CD56+ cells exert antitumor activity via antigen specific and antigen nonspecific mechanisms (14) (15). Elevated circulating CD19+ lymphocytes can predict survival in patients with gastric cancer (16). There have been many other studies which have shown CD3+, CD4+, CD8+, CD19+, and CD56+ subsets are important in antitumor immunity, and immune suppression may increase the risk of tumor growth and metastasis (17) (18) (19) (20) (21) (22). Therefore, the reduction of RT induced suppression of these lymphocyte subsets has the potential for decreasing tumor growth and metastasis.

Circulating lymphocytes are highly radiosensitive and TRL is currently considered an unavoidable side-effect of RT. Optimizing RT treatment planning to reduce lymphopenia by considering circulating blood as a critical Organ At Risk (OAR) has not been extensively studied. Current national Radiation Therapy protocols are oblivious to lymphopenia.

Currently, there are no models to accurately predict lymphocyte loss for patients undergoing radiation therapy. Doses to different organs affect the lymphocyte depletion in different ways. The maximum dose and the mean dose for a radiation plan are not necessarily the best parameters for evaluating the level of lymphopenia. Instead, the dynamics between the time dependent dose to structures and velocities of blood through those organs need to be carefully taken into account to predict the expected lymphocyte loss, also called lymphocyte kill, lymph kill, or cell kill, for a given RT plan. Therefore, it is not possible to determine the lymph kill level of a plan by using the optimization parameters currently available in treatment planning systems.

The present invention is the first algorithm for predicting post-treatment time dependent lymphocyte counts for RT treatments. In addition to predictive capacity for individual patients, this algorithm can provide important parameters such as a dose dependent lymphocyte kill function and average net release rate of new lymphocytes into circulating blood which also includes the proliferation of existing cells and natural death of lymphocytes in blood. The results were compared to measurements to quantify the predictability of the algorithm. This predictive algorithm will enable treatment plan design and optimization to give the lowest possible TRL, while maintaining all other current clinical dosimetric requirements.

### SUMMARY OF ASPECTS OF EMBODIMENTS OF THE PRESENT INVENTION

The system, method and computer readable medium to estimate the post-treatment blood cell sub type count in patients treated via radiation therapy are an extension, and an improvement, to the published work of S. Yuvino, et al (23) which looked at the brain as a homogeneous organ. A program was written in MATLAB (The Mathworks, Natick, Mass.) to simulate the delivery of the radiation dose to moving blood in the body. For each patient, radiation therapy treatment plans, CT planning image sets, including contoured structure sets, dose maps per each treatment field, structure sets, and delivery times are accessed and imported into the simulation model via DICOM (Digital Imaging and Communications in Medicine) import to obtain the time dependent organ specific doses for each voxel of that organ. This, combined with a blood flow model (with organ dependent cardiac outputs and blood velocities) for each organ within the simulation is used to calculate absorbed dose values, Di, for the circulating blood/lymphocyte population. These absorbed doses are used to predict a lymphocyte kill fraction, using a time dependent fractional blood count, by incorporating a dose dependent cell survival curve. The kill probability function for a lymphocyte, K(D), depends on the dose absorbed by the lymphocyte, D. Finally, the remaining lymphocyte count, N(t), at a time, t, following RT is calculated by addition of a time dependent net release rate of new lymphocytes to the circulating blood, R(N₀-N(t)), to represent the combined effects of release from the lymphoid organs to blood, as well as a proliferation of the existing cells, and natural death of lymphocytes in blood. The following parameterization summarizes the model for the lymphocyte level as a function of time, N(t):

$$N ( t ) = {N}_{0} {\sum }_{i = 0}^{i = {N}_{0}} [ 1 - K ( {D}_{i} ) ] \text{/} {N}_{0} + R ( {N}_{0} - N ( t ) ) \cdot t \qquad ( 1 )$$

An aspect of an embodiment of the present invention provides, among other things, a predictive algorithm (method and technique) to estimate the post-treatment blood cell sub-type count in patients treated via radiation therapy.

An aspect of an embodiment of the present invention provides a system, method and computer readable medium for, among other things, treatment related lymphopenia in lung SBRT—with clinical relevance and a predictive model.

An aspect of an embodiment of the present invention provides, among other things, a predictive algorithm (method and technique) of post-treatment lymphocyte count in patients treated via radiation therapy.

An aspect of various embodiments of the present invention may provide a number of novel and nonobvious features, elements and characteristics, such as but not limited thereto, the following:
1. a system, method and computer readable medium for modeling a prediction of post-treatment lymphocyte drop, also called lymphocyte kill, lymph kill, or cell kill;
2. a system, method and computer readable medium to account for all organs in the body, and consider the circulating lymphocytes and interplay between the time dependent dose and the blood circulation time;
3. a system, method and computer readable medium for modeling blood flow through organs with different velocities without leaking in to other organs;
4. a system, method and computer readable medium for modeling that may utilize cell kill models available to create the lymphocyte kill due to the dose absorption;
5. a system, method and computer readable medium that provides a model to also include a function to account for regeneration, natural repopulation of the lymphocytes, and natural death of lymphocytes in blood;
6. a system, method and computer readable medium that provides a model that has been tested with real patient lymphocyte drop and has a high predictability (sensitivity and specificity high); and
7. a system, method and computer readable medium for modeling a prediction of post-treatment lymphocyte sub-population count (T cells such as CD3+. CD4+, CD8+, CD19+, CD56+, . . . ).

An aspect of an embodiment of the present invention provides, among other things, a system for use in estimating the post-treatment blood cell sub type count of a subject treated via radiation therapy. The system may comprise: a computer processor; a memory configured to store instructions that are executable by the computer processor, wherein the computer processor is configured to execute the instructions for: performing processing associated with importing subject data into a simulation model; performing processing associated with determining at least one time dependent dose for each voxel of at least one organ of the subject within the simulation model; performing processing associated with creating a blood flow model for the at least one organ of the subject within the simulation model; performing processing associated with simulating the delivery of a radiation dose to moving blood within the subject's body within the simulation model using the at least one time dependent dose for each voxel of the at least one organ of the subject and the blood flow model; performing processing associated with determining at least one absorbed dose value for the subject's blood cell sub type within the simulation model; performing processing associated with calculating a remaining blood cell sub type count; and performing processing associated with transmitting the remaining blood cell sub type count to a secondary source.

An aspect of an embodiment of the present invention provides, among other things, a computer method for estimating the post-treatment blood cell sub type count of a subject treated via radiation therapy. The method may comprise: performing processing associated with importing subject into a simulation model; performing processing associated with determining at least one time dependent dose for each voxel of at least one organ of the subject within the simulation model; performing processing associated with creating a blood flow model for the at least one organ of the subject within the simulation; performing processing associated with simulating the delivery of a radiation dose to moving blood within the subject's body within the simulation model using the at least one time dependent dose for each voxel of the at least one organ of the subject and the blood flow model; performing processing associated with determining at least one absorbed dose value for the subject's blood cell sub type within the simulation model; performing processing associated with calculating a remaining blood cell sub type count; and performing processing associated with transmitting the remaining blood cell sub type count to a secondary source.

An aspect of an embodiment of the present invention provides, among other things, a non-transitory, computer readable storage medium having instructions stored thereon for use in estimating the post-treatment blood cell sub type count of a subject treated via radiation therapy that, when executed by a computer processor, cause the computer processor to: receive subject data for a simulation model; determine at least one time dependent dose for each voxel of at least one organ of the subject within the simulation model; create a blood flow model for the at least one organ of the subject within the simulation model; simulate the delivery of a radiation dose to moving blood within the subject's body using the at least one time dependent dose for each voxel of the at least one organ of the subject within the simulation model and the blood flow model; determine at least one absorbed dose value for the subject's blood cell sub type; calculate a remaining blood cell sub type count within the simulation model; and transmit the remaining blood cell sub type count to a secondary source.

An aspect of an embodiment of the present invention provides, among other things, a system, method, and computer readable medium for estimating the patient specific and plan specific radiation dose delivered to any type of circulating blood cell type or sub-type, such as, but not limited to, T lymphocytes, B lymphocytes, natural killer cells, erythrocytes, or neutrophils, and predicting time dependent fractional blood count and cell kill following radiation therapy treatment. Additionally, the system, method, and computer readable medium provide parameters such as a dose dependent lymphocyte kill function and average net release rate of new lymphocytes into circulating blood, which also includes the proliferation of existing cells and natural death of lymphocytes in blood. Determining lymphocyte kill following Stereotactic Body radiation therapy (SBRT) to lung tumors is an example of an application of the system, method, and computer readable medium.

The invention itself, together with further objects and attendant advantages, will best be understood by reference to the following detailed description, taken in conjunction with the accompanying drawings.

These and other objects, along with advantages and features of various aspects of embodiments of the invention disclosed herein, will be made more apparent from the description, drawings and claims that follow.

### BRIEF DESCRIPTION OF THE DRAWINGS

The foregoing and other objects, features and advantages of the present invention, as well as the invention itself, will be more fully understood from the following description of preferred embodiments, when read together with the accompanying drawings

The accompanying drawings, which are incorporated into and form a part of the instant specification, illustrate several aspects and embodiments of the present invention and, together with the description herein, serve to explain the principles of the invention. The drawings are provided only for the purpose of illustrating select embodiments of the invention and are not to be construed as limiting the invention.

FIG. 1 is a graphical representation demonstrating the relationship between survival and grade III/IV TRL and the association between severe TRL in 40% of patients two months after the initiation of chemoradiation with shorter survival from tumor progression.

FIG. 2 is a graphical representation demonstrating a statistically significant survival difference for patients with higher TLC.

FIG. 3 is a flowchart demonstrating the general procedure for calculating the dose delivered to blood flowing through an organ.

FIG. 4 is a block diagram illustrating an example of a machine upon which one or more aspects of embodiments of the present invention can be implemented.

FIG. 5 is a flowchart demonstrating modeling blood flow through organs without leakage.

FIG. 6(A) is a simulated diagram of the superior view [towards the head] from a transverse or axial [horizontal plane dividing top and bottom] cross-section of a subject, with simulated organs showing the inclusion of all organs in the thorax.

FIG. 6(B) is a simulated diagram of the medial view [towards the middle] from a sagittal or mid-sagittal [longitudinal, vertical plane dividing left and right] cross-section of a subject, with simulated organs showing the inclusion of all organs in the thorax as well as the flow of blood through the blood rich organs.

FIG. 6(C) is a simulated diagram of the anterior view from a frontal or coronal [vertical plane dividing front and back] cross-section of a subject, with simulated organs showing the inclusion of all organs in the thorax as well as the flow of blood through the blood rich organs.

FIG. 7 is a simulated diagram of generated aortal dose snapshots for one subject at different time points during lung SBRT treatment. Darkest gray is no dose, and it shifts to lightest gray (3Gy) as the dose increases. Percentage of total blood volume that has accumulated more than 3Gy circulation through the body at the end of different days were: t=0: 0%, t=240 s: 0.4%, t=720 s: 1.2%, t=1200 s: 2.1.

FIG. 8 is a graphical representation comparing the generated survival fractions (1-K(D)) for a subject to Nakamura et al (27) measured data points.

FIG. 9(A) is a graphical representation demonstrating the simulation predicted and measured post treatment LYA count as a function of pre-treatment LYA count.

FIG. 9(B) is a graphical representation demonstrating the LYA difference between simulation and measurement as a function of pre-treatment LYA value.

FIG. 10 is a graphical representation demonstrating the ROC analysis using the MATLAB perfcurve function for a prediction of post treatment LYA count to be <0.8×10⁹ cells/L.

FIG. 11(A) is a graphical representation showing an example distribution of absorbed dose to blood cells from a single subject.

FIG. 11(B) is a graphical representation showing the associated percentage kill contribution for a single patient.

FIG. 11(C) is a graphical representation showing the kill contribution average across all subjects.

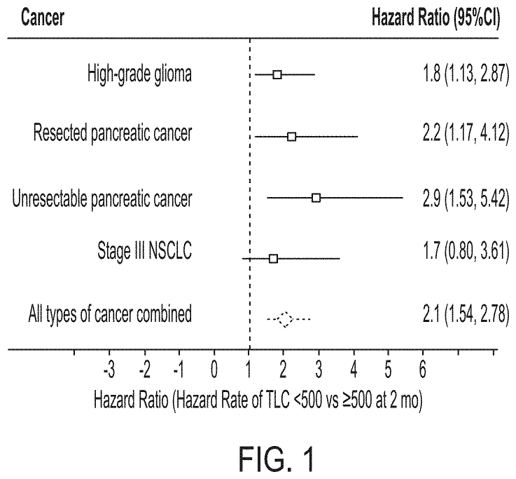

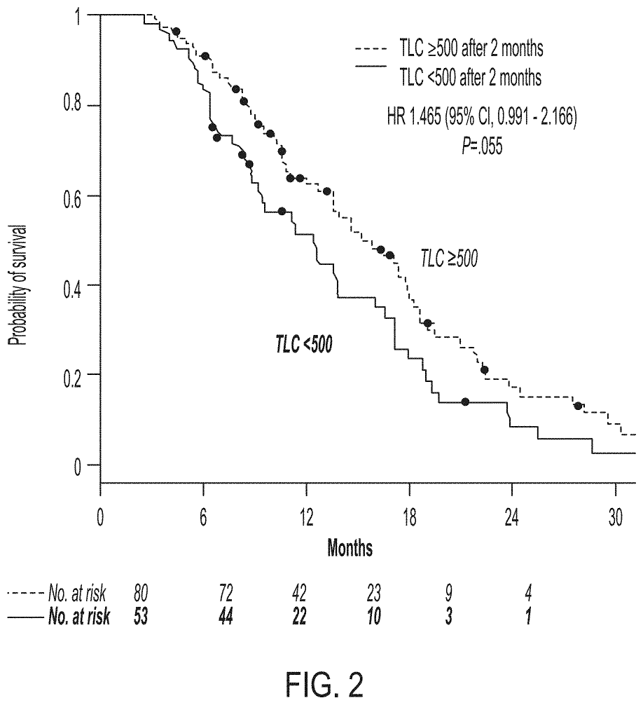

### DETAILED DESCRIPTION OF EXEMPLARY EMBODIMENTS

The system, method and computer readable medium to estimate the post-treatment blood cell sub-type count in patients treated via radiation therapy may be used for, among other things, predicting post-treatment time dependent lymphocyte counts for RT treatments.

### General Simulation Procedure for an Embodiment of the Invention

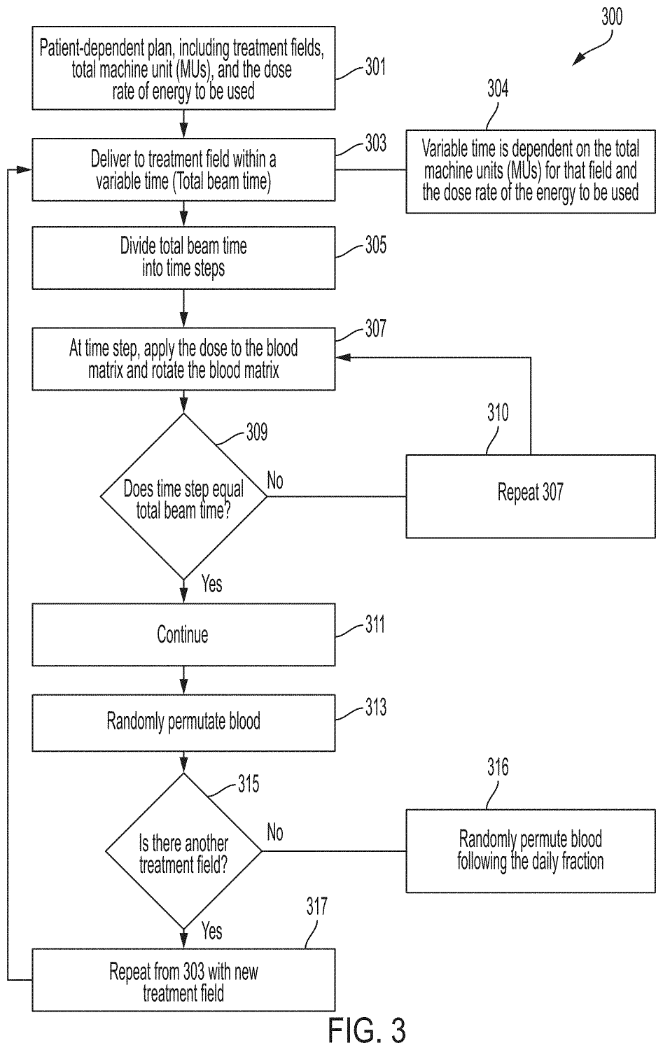

Radiation dose to circulating blood cells is calculated using the very few assumptions: (i) total blood volume was assumed to be 5 L (24) and (ii) the heart-to-heart blood circulation time was assumed to be 30 seconds. In an embodiment, the heart-to-heart blood circulation time is chosen from within the following range: about 10 to about 50 seconds. All other parameters are calculated in a patient-dependent and plan-dependent fashion, as explained below and by FIG. 3, which is a flowchart 300 demonstrating the general procedure for calculating the dose delivered to blood flowing through an organ. It should be appreciated that, in an embodiment, the general procedure of the flowchart 300 shown in FIG. 3 may calculate the dose delivered to blood flowing through more than one organ.

A) Treatment delivery time or treatment beam delivery time:

Starting with FIG. 3, a patient-dependent plan 301 provides data including, but not limited to, treatment fields, total machine units (MUs), and the dose rate of energy to be used. It should be appreciated that the patient-dependent plan 301 also includes other relevant patient or subject data either explicitly or inherently derived from the patient-dependent plan. Types of patient-dependent plans 301 may include, but are not limited to, radiation therapy treatment plans, molecular imaging planning image sets, dose maps, contoured or uncontoured structure sets, and/or delivery times. Patient-dependent plan 301 may also include blood cell sub type distribution(s), pre-treatment rate of regeneration, pre-treatment rate of redistribution, or subject age subject data. Each treatment field is delivered within a variable time 303 depending on the total delivered machine units (MUs) for that field and the dose rate of the energy used 304 as provided by the patient-dependent plan 301.

B) Time dependent dose delivered to each voxel of a blood flow model to determine the absorbed dose values:

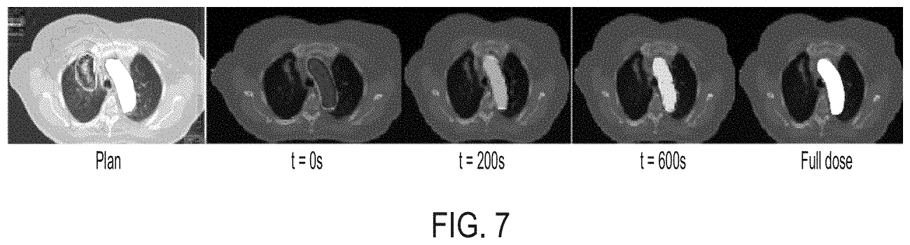

FIG. 3, illustrating a flowchart 300, involves breaking the total beam time into time steps 305. At each time step, the dose is applied to the voxels of the blood matrix and the blood matrix is rotated 307 to simulate blood flow through the organ. The time step is determined 309. If the time step does not equal the total beam time 310, the dose is applied to the blood matrix and the blood matrix is rotated 307 until the time step equals the total beam time 311. Following each treatment field, blood is randomly permuted 313 to simulate mixing with the remaining blood in the rest of the body. It is determined whether there are any other treatment fields 315. If no, the blood is randomly permuted following the daily fraction 316. If yes, the process is repeated with the new treatment field 317. Once all treatment fields are completed, the blood is randomly permuted following the daily fraction 316 and the absorbed dose values for each voxel are stored in the blood matrix. FIG. 7 shows simulation generated aortal dose snapshots for one patient case at different time steps (t) during lung SBRT treatment. Darkest gray, at t=0 seconds, is no dose, and it shifts to lightest gray, full dose (3Gy) at total beam time as the dose increases. Percentage of total blood volume that has accumulated more than 3Gy circulation through the body at the end of different days/daily fractions were: t=0: 0%, t=240 s: 0.4%, t=720 s: 1.2%, t=1200 s: 2.1.

C) Modeling blood flow through organs without leakage:

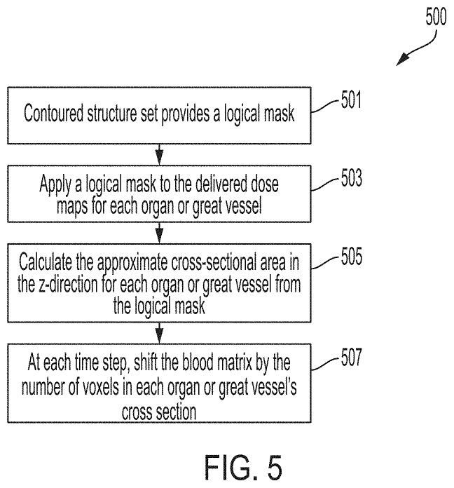

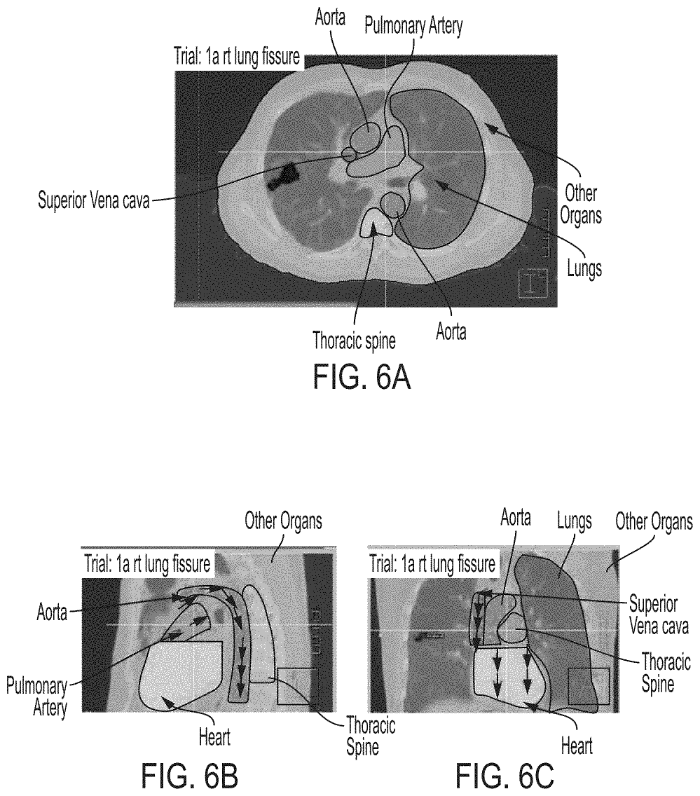

FIG. 5 demonstrates modeling blood flow through organs without leakage by a flowchart 500. Logical masks are provided in the contoured structure set(s) 501. In an embodiment, logical masks may be applied without having been provided in the contoured structure set(s). The dose delivered to great vessels and other major organs in the field is obtained by applying a logical mask to the patient-dependent delivered dose maps for each organ or great vessel 503 from the patient dependent plan 301. By using logical masks for each organ or great vessel, the dose for each of these structures is applied while avoiding leakage into other organs. For each organ, the approximate cross-sectional area in the z-direction is calculated from the logical mask 505 and used to determine how much to shift the blood matrix at each time step 507. At each time step, the blood matrix is shifted by the number of voxels in the organ or great vessel's cross section 507, in order to simulate blood flow through the organ. This use of cross-sectional area provides a method to simulate blood flow through complex organ and/or great vessel shapes while ensuring good accuracy over the course of an entire treatment. This separation of organ and/or great vessels is done for the great vessels such as the aorta, vena cava, and pulmonary artery, as well as major organs in the treatment field for each patient (such as the liver, heart, lungs, or stomach). For example, FIG. 6(A) shows the superior view [towards the head] from a transverse or axial [horizontal plane dividing top and bottom] cross-section of a subject, with simulated organs showing the inclusion of all organs in the thorax, FIG. 6(B) shows the medial view [towards the middle] from a sagittal or mid-sagittal [longitudinal, vertical plane dividing left and right] cross-section of a subject, with simulated organs showing the inclusion of all organs in the thorax as well as the flow of blood through the blood rich organs, and FIG. 6(C) shows the anterior view from a frontal or coronal [vertical plane dividing front and back] cross-section of a subject, with simulated organs showing the inclusion of all organs in the thorax as well as the flow of blood through the blood rich organs.

Separating doses to circulatory and other organs also allows differences in blood density and velocity across organs to be accounted for. In an embodiment, the average blood density per voxel is determined by dividing the total blood volume of 5 L by the number of voxels in the body. In other embodiments, the total blood volume is chosen from the following range: about 2 L to about 7 L. In an embodiment, it is assumed that the volume covered in the CT image is approximately one third of the total body. The blood flow rate in each organ, in units of cross-sectional layers per second, is determined using the following formula:

$$v = \frac{\frac{\frac{5 Liters}{30 seconds} * CO * 1 layer}{cvoxels} * totalvoxels}{5 Liters}$$

Here, c is the number of voxels in one cross sectional layer, and CO is the cardiac output of the given organ, given by Table 17 in (25). For great vessels, the cardiac output is 100% and the result, v, is multiplied by an additional factor, gv, which accounts for a higher blood density flowing through great vessels. In an embodiment, a value of 8 is chosen for gv, which gives great vessel blood velocities around 11 cm/s and the total blood volume in the heart at any given time is around 300 mL, in accordance with existing literature (26).

In an embodiment, during the simulation for each organ or great vessel, the blood matrix is rotated by one layer every 1/v seconds. The dose to the stationary lymphocytes in the thoracic spine is estimated by separating the dose to the spine and setting the blood velocity to zero.

Review paper (25) analyzes data on blood flow and identifies representative percentages of cardiac output to different organs. This publication also provides absolute blood flow rates to organs and tissue types of humans. Total blood volume as a function of patient height, weight, sex, and age is given in Ref (24).

In an embodiment, the blood velocities for an example patient are calculated and compared to published data (26) in Table I. For the vena cava for many patients, the small size of the organ coupled with the high rate of blood flow through the organ results in very high blood velocities. This is because the assumptions made for how blood flows through the organs apply best when the organs are large and deviations can be balanced out. For very small high-flow organs like the vena cava, the assumptions break down and give unrealistic speeds. For this reason, blood flow is capped at 25.0 cm/s through these organs.

TABLE I

| | | Blood Velocity |
|---|---|---|
| | Blood Velocity | published (cm/s) |
| Organ | calculated (cm/s) | Average, peak |
| Lungs | 0.6 | 0.5-1 cm/s (inferred) |
| Aorta | 15.0 | 11 cm/s, 66 cm/s |
| Pulmonary Artery | 13.0 | 10 cm/s, 57 cm/s |
| Vena Cava | 25.0 | Superior: 12 cm/s, 28 cm/s |
| | | Inferior: 13 cm/s, 26cm/s |

In an embodiment, to better match results from published literature, we use a higher blood density in great vessels than for other organs. The implementation of this increased density is discussed above. The choice of density factor gv=8 gave a blood volume of approximately 350 mL in the heart. This is in rough agreement with existing literature, which reports a heart blood volume of 300 mL.

### Cell Kill Models

Nakamura et al (27) demonstrated that a dose of 0.5 Gy kills 10% of the lymphocytes, 2 Gy kills 50% of the lymphocytes, and 3 Gy kills approximately 90% of lymphocytes, and published a linear quadratic (LQ) model to fit this data. In addition, it has been reported that 0.5 Gy is a threshold for lymphocyte kill (28). In equation 1, we employ three different lymphocyte kill function models, K(Dᵢ), also called kill probability functions or models. Since most of these models are based on in vitro data, and carry an inherent inaccuracy when dealing with cell kill within humans, in an embodiment, in addition to incorporating the cell kill model given by Ref (27), we optimize the algorithm to fit our observed data within each cell kill parameterization.

Once the cumulative blood dose, also called the absorbed dose value, is calculated and stored in the blood voxel matrix, the fractional lymphocyte kill, and thereby remaining blood cell sub type count, could be estimated. Three different lymphocyte kill functions, or kill probability functions, K(Dᵢ), are used to calculate lymphocyte kill given the dose to a given blood voxel. For each kill function, the total blood kill is calculated by summing the kill function values for each voxel to determine the blood kill percentage, which is then multiplied by the initial LYA count (initial blood cell sub type distribution) to obtain the absolute LYA reduction.

a) The first model is an exponential function using the linear-quadratic (LQ) model (29), of the form:

K(D)=1−exp(−αD−βD²).

In an embodiment, the two parameters, α and β, are determined using the fixed condition that K(5Gy)=0.992 as given by Nakamura et al. (27), and the value K(0.5Gy), which was left free to vary.

b) The second model is a fractionated version of the linear quadratic model (30). The kill is calculated based on the doses delivered after one fraction (d), according to the function:

K(D)=1−exp(−nd(α+βd))

In an embodiment, the parameters α and β here are determined using the same conditions as those for the first kill function.

c) The third model was a point-to-point spline fit to the data points presented in Nakamura (27): K(2Gy)=0.65, K(3Gy)=0.88, K(4Gy)=0.97, K(5Gy)=0.992. Furthermore, between data points, n is equal to the value of the first data point. In an embodiment, an additional spline point for K(0.5Gy) is allowed to vary. Between each data point n=0 to 5, pₙ ∈ [0,0.5,2,3,4,5], the kill function was equal to:

$$K ( D ) = \frac{K ( {p}_{n + 1} ) - K ( {p}_{n} )}{{p}_{n + 1} - {p}_{n}} ( D - {p}_{n} ) + K ( {p}_{n} )$$

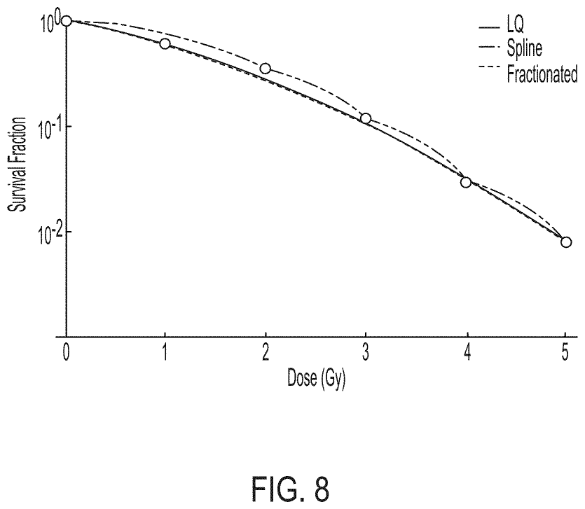

FIG. 8 graphically demonstrates a simulation generated survival fraction (1-K(D)), the measurement of lymphocyte survival after lymphocyte kill, which is also called lymph kill or cell kill, using each kill function and compared to measured data points from Nakamura (27) represented by dots.

In our study, we expected that the majority of blood cells would receive around 0.5 Gy during the treatment. Therefore, in an embodiment, we choose to allow the 0.5 Gy data point to vary during fitting for all models. In the optimization process, the χ² difference between the simulated lymph kill according to the new function and the measured LYA reduction is calculated and minimized using the finin library in MATLAB.

### Future Improvements to the Model

The simulation assumes a constant net release rate of new lymphocytes to the circulating blood to represent the combined effects of release from the lymphoid organs to blood, as well as a proliferation of the existing cells and the natural death of lymphocytes in blood. It has been observed (31) that lymphocyte kill peaks at 25 days after first day of the SBRT treatment for lung.

It has been shown that lymphocyte counts, sub-counts, and their proliferative capacity change with patient's age. Each part of the immune system is influenced to some extent by the aging process (32) (33) (34). Furthermore, there is a possibility that when the circulating T cells are killed in RT, there is no quick feedback mechanism to replenish them from the lymphoid organs in older patients. In order to account for the age-related effects on lymphocyte counts and proliferation in patients, an embodiment can define age and pre-treatment lymphocyte count dependent replenishment rates. In another embodiment, age and pre-treatment cell count dependent replenishment rates can be defined for other cell types or sub-types, including but not limited to: lymphocyte sub-types, natural killer cells, erythrocytes, or neutrophils.

In an embodiment, the model will include the fraction of local/stationary lymphocytes in different organs and vessels.

In an embodiment, the model will include the variation of blood velocities from the center to the wall of great vessels as described in (26).

In an embodiment, different sub types of lymphocytes will be assigned a different sensitivity to a given dose.

In an embodiment, regeneration and redistribution will be modeled as a function of pre-treatment lymphocyte count as well as age of the patient.

By performing a well-planned clinical trial, we plan to create a cleaner input data dataset that has the ability to refine these issues in the model such as rate of regeneration. This clinical trial, using IGRT will ensure not only the location of the tumor, but also the reproducibility of blood rich organs with the treatment plan on a daily basis.

### Conclusion

We have developed a predictive model to evaluate the post treatment lymphocyte counts following lung SBRT. This model includes critical elements: a) lymphocyte distribution, b) interaction between RT delivery and the blood transport system, c) the cell survival with radiation, d) cell regeneration model and e) blood volumes, cardiac output, blood velocity, and treatment delivery time. Like most algorithms, our algorithm relies on widely accepted assumptions such as the cell survival model with radiation. These elements are strongly supported by our preliminary results: a) predict the post treatment absolute lymphocyte value to better than 16% across all variables of interest: age, pre-treatment LYA value (initial blood cell sub type distribution), post-treatment blood draw day, treatment delivery time, tumor volume size, and location of the tumor and b) our current preliminary model has a sensitivity and a specificity to predict a patient having a post RT lymphocyte value of <0.8×10⁹ cells/L with an area under the curve (AUC) of Receiver Operating Characteristic (ROC) of 0.84.

This model could be used to predict the radiation related cell kill of any type of circulating blood cell. It should be appreciated that, while lymphocyte kill is used as an example embodiment, other cell types or sub-types may be used in other embodiments, including but not limited to: lymphocyte sub-types, natural killer cells, erythrocytes, or neutrophils.

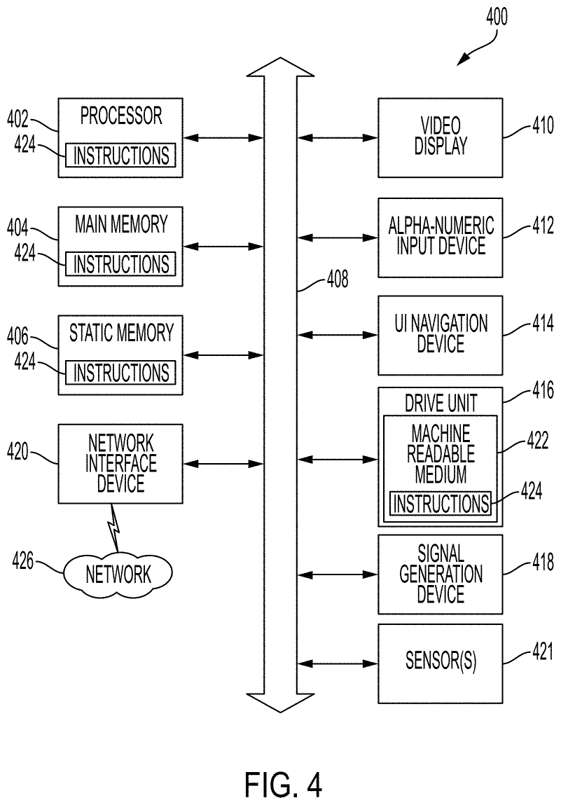

FIG. 4 An aspect of an embodiment of the present invention includes, but is not limited thereto, a system, method, and computer readable medium that provides: a) predictive technique to estimate the post-treatment blood cell sub-type count in patients treated via radiation therapy, b) treatment related lymphopenia in lung SBRT—with clinical relevance and a predictive model, and/or c) post-treatment lymphocyte count in patients treated via radiation therapy, which illustrates a block diagram of an example machine 400 upon which one or more embodiments (e.g., discussed methodologies) can be implemented (e.g., run).

Examples of machine 400 can include logic, one or more components, circuits (e.g., modules), or mechanisms. Circuits are tangible entities configured to perform certain operations. In an example, circuits can be arranged (e.g., internally or with respect to external entities such as other circuits) in a specified manner. In an example, one or more computer systems (e.g., a standalone, client or server computer system) or one or more hardware processors (processors) can be configured by software (e.g., instructions, an application portion, or an application) as a circuit that operates to perform certain operations as described herein. In an example, the software can reside (1) on a non-transitory machine readable medium or (2) in a transmission signal. In an example, the software, when executed by the underlying hardware of the circuit, causes the circuit to perform the certain operations.

In an example, a circuit can be implemented mechanically or electronically. For example, a circuit can comprise dedicated circuitry or logic that is specifically configured to perform one or more techniques such as discussed above, such as including a special-purpose processor, a field programmable gate array (FPGA) or an application-specific integrated circuit (ASIC). In an example, a circuit can comprise programmable logic (e.g., circuitry, as encompassed within a general-purpose processor or other programmable processor) that can be temporarily configured (e.g., by software) to perform the certain operations. It will be appreciated that the decision to implement a circuit mechanically (e.g., in dedicated and permanently configured circuitry), or in temporarily configured circuitry (e.g., configured by software) can be driven by cost and time considerations.

Accordingly, the term “circuit” is understood to encompass a tangible entity, be that an entity that is physically constructed, permanently configured (e.g., hardwired), or temporarily (e.g., transitorily) configured (e.g., programmed) to operate in a specified manner or to perform specified operations. In an example, given a plurality of temporarily configured circuits, each of the circuits need not be configured or instantiated at any one instance in time. For example, where the circuits comprise a general-purpose processor configured via software, the general-purpose processor can be configured as respective different circuits at different times. Software can accordingly configure a processor, for example, to constitute a particular circuit at one instance of time and to constitute a different circuit at a different instance of time.

In an example, circuits can provide information to, and receive information from, other circuits. In this example, the circuits can be regarded as being communicatively coupled to one or more other circuits. Where multiple of such circuits exist contemporaneously, communications can be achieved through signal transmission (e.g., over appropriate circuits and buses) that connect the circuits. In embodiments in which multiple circuits are configured or instantiated at different times, communications between such circuits can be achieved, for example, through the storage and retrieval of information in memory structures to which the multiple circuits have access. For example, one circuit can perform an operation and store the output of that operation in a memory device to which it is communicatively coupled. A further circuit can then, at a later time, access the memory device to retrieve and process the stored output. In an example, circuits can be configured to initiate or receive communications with input or output devices and can operate on a resource (e.g., a collection of information).

The various operations of method examples described herein can be performed, at least partially, by one or more processors that are temporarily configured (e.g., by software) or permanently configured to perform the relevant operations. Whether temporarily or permanently configured, such processors can constitute processor-implemented circuits that operate to perform one or more operations or functions. In an example, the circuits referred to herein can comprise processor-implemented circuits.

Similarly, the methods described herein can be at least partially processor-implemented. For example, at least some of the operations of a method can be performed by one or processors or processor-implemented circuits. The performance of certain of the operations can be distributed among the one or more processors, not only residing within a single machine, but deployed across a number of machines. In an example, the processor or processors can be located in a single location (e.g., within a home environment, an office environment or as a server farm), while in other examples the processors can be distributed across a number of locations.

The one or more processors can also operate to support performance of the relevant operations in a “cloud computing” environment or as a “software as a service” (SaaS). For example, at least some of the operations can be performed by a group of computers (as examples of machines including processors), with these operations being accessible via a network (e.g., the Internet) and via one or more appropriate interfaces (e.g., Application Program Interfaces (APIs)).

Example embodiments (e.g., apparatus, systems, or methods) can be implemented in digital electronic circuitry, in computer hardware, in firmware, in software, or in any combination thereof. Example embodiments can be implemented using a computer program product (e.g., a computer program, tangibly embodied in an information carrier or in a machine readable medium, for execution by, or to control the operation of, data processing apparatus such as a programmable processor, a computer, or multiple computers).

A computer program can be written in any form of programming language, including compiled or interpreted languages, and it can be deployed in any form, including as a stand-alone program or as a software module, subroutine, or other unit suitable for use in a computing environment. A computer program can be deployed to be executed on one computer or on multiple computers at one site or distributed across multiple sites and interconnected by a communication network.

In an example, operations can be performed by one or more programmable processors executing a computer program to perform functions by operating on input data and generating output. Examples of method operations can also be performed by, and example apparatus can be implemented as, special purpose logic circuitry (e.g., a field programmable gate array (FPGA) or an application-specific integrated circuit (ASIC)).

The computing system can include clients and servers. A client and server are generally remote from each other and generally interact through a communication network. The relationship of client and server arises by virtue of computer programs running on the respective computers and having a client-server relationship to each other. In embodiments deploying a programmable computing system, it will be appreciated that both hardware and software architectures require consideration. Specifically, it will be appreciated that the choice of whether to implement certain functionality in permanently configured hardware (e.g., an ASIC), in temporarily configured hardware (e.g., a combination of software and a programmable processor), or a combination of permanently and temporarily configured hardware can be a design choice. Below are set out hardware (e.g., machine 400) and software architectures that can be deployed in example embodiments.

In an example, the machine 400 can operate as a standalone device or the machine 400 can be connected (e.g., networked) to other machines.

In a networked deployment, the machine 400 can operate in the capacity of either a server or a client machine in server-client network environments. In an example, machine 400 can act as a peer machine in peer-to-peer (or other distributed) network environments. The machine 400 can be a personal computer (PC), a tablet PC, a set-top box (STB), a Personal Digital Assistant (PDA), a mobile telephone, a web appliance, a network router, switch or bridge, or any machine capable of executing instructions (sequential or otherwise) specifying actions to be taken (e.g., performed) by the machine 400. Further, while only a single machine 400 is illustrated, the term “machine” shall also be taken to include any collection of machines that individually or jointly execute a set (or multiple sets) of instructions to perform any one or more of the methodologies discussed herein.

Example machine (e.g., computer system) 400 can include a processor 402 (e.g., a central processing unit (CPU), a graphics processing unit (GPU) or both), a main memory 404 and a static memory 406, some or all of which can communicate with each other via a bus 408. The machine 400 can further include a display unit 410, an alphanumeric input device 412 (e.g., a keyboard), and a user interface (UI) navigation device 411 (e.g., a mouse). In an example, the display unit 810, input device 417 and UI navigation device 414 can be a touch screen display. The machine 400 can additionally include a storage device (e.g., drive unit) 416, a signal generation device 418 (e.g., a speaker), a network interface device 420, and one or more sensors 421, such as a global positioning system (GPS) sensor, compass, accelerometer, or other sensor.

The storage device 416 can include a machine readable medium 422 on which is stored one or more sets of data structures or instructions 424 (e.g., software) embodying or utilized by any one or more of the methodologies or functions described herein. The instructions 424 can also reside, completely or at least partially, within the main memory 404, within static memory 406, or within the processor 402 during execution thereof by the machine 400. In an example, one or any combination of the processor 402, the main memory 404, the static memory 406, or the storage device 416 can constitute machine readable media.

While the machine readable medium 422 is illustrated as a single medium, the term “machine readable medium” can include a single medium or multiple media (e.g., a centralized or distributed database, and/or associated caches and servers) that configured to store the one or more instructions 424. The term “machine readable medium” can also be taken to include any tangible medium that is capable of storing, encoding, or carrying instructions for execution by the machine and that cause the machine to perform any one or more of the methodologies of the present disclosure or that is capable of storing, encoding or carrying data structures utilized by or associated with such instructions. The term “machine readable medium” can accordingly be taken to include, but not be limited to, solid-state memories, and optical and magnetic media. Specific examples of machine readable media can include non-volatile memory, including, by way of example, semiconductor memory devices (e.g., Electrically Programmable Read-Only Memory (EPROM), Electrically Erasable Programmable Read-Only Memory (EEPROM)) and flash memory devices; magnetic disks such as internal hard disks and removable disks; magneto-optical disks; and CD-ROM and DVD-ROM disks.

The instructions 424 can further be transmitted or received over a communications network 426 using a transmission medium via the network interface device 420 utilizing any one of a number of transfer protocols (e.g., frame relay, IP, TCP, UDP, HTTP, etc.). Example communication networks can include a local area network (LAN), a wide area network (WAN), a packet data network (e.g., the Internet), mobile telephone networks (e.g., cellular networks), Plain Old Telephone (POTS) networks, and wireless data networks (e.g., IEEE 802.11 standards family known as Wi-Fi®, IEEE 802.16 standards family known as WiMax®), peer-to-peer (P2P) networks, among others. The term “transmission medium” shall be taken to include any intangible medium that is capable of storing, encoding or carrying instructions for execution by the machine, and includes digital or analog communications signals or other intangible medium to facilitate communication of such software.

Moreover, it should be appreciated that any of the components or modules referred to with regards to any of the present invention embodiments discussed herein, may be integrally or separately formed with one another. Further, redundant functions or structures of the components or modules may be implemented. Moreover, the various components may be communicated locally and/or remotely with any user or machine/system/computer/processor. Moreover, the various components may be in communication via wireless and/or hardwire or other desirable and available communication means, systems and hardware. Moreover, various components and modules may be substituted with other modules or components that provide similar functions.

It should be appreciated that the device and related components discussed herein may take on all shapes along the entire continual geometric spectrum of manipulation of x, y and z planes to provide and meet the environmental, anatomical, and structural demands and operational requirements. Moreover, locations and alignments of the various components may vary as desired or required.

It should be appreciated that various sizes, dimensions, contours, rigidity, shapes, flexibility and materials of any of the components or portions of components in the various embodiments discussed throughout may be varied and utilized as desired or required.

It should be appreciated that while some dimensions are provided on the aforementioned figures, the device may constitute various sizes, dimensions, contours, rigidity, shapes, flexibility and materials as it pertains to the components or portions of components of the device, and therefore may be varied and utilized as desired or required.

Although example embodiments of the present disclosure are explained in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

It must also be noted that, as used in the specification and the appended claims, the singular forms “a,” “an” and “the” include plural referents unless the context clearly dictates otherwise. Ranges may be expressed herein as from “about” or “approximately” one particular value and/or to “about” or “approximately” another particular value. When such a range is expressed, other exemplary embodiments include from the one particular value and/or to the other particular value.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

In describing example embodiments, terminology will be resorted to for the sake of clarity. It is intended that each term contemplates its broadest meaning as understood by those skilled in the art and includes all technical equivalents that operate in a similar manner to accomplish a similar purpose. It is also to be understood that the mention of one or more steps of a method does not preclude the presence of additional method steps or intervening method steps between those steps expressly identified. Steps of a method may be performed in a different order than those described herein without departing from the scope of the present disclosure. Similarly, it is also to be understood that the mention of one or more components in a device or system does not preclude the presence of additional components or intervening components between those components expressly identified.

Some references, which may include various patents, patent applications, and publications, are cited in a reference list and discussed in the disclosure provided herein.

The citation and/or discussion of such references is provided merely to clarify the description of the present disclosure and is not an admission that any such reference is “prior art” to any aspects of the present disclosure described herein. In terms of notation, “(n)” corresponds to the n^{th} reference in the list. All references cited and discussed in this specification are incorporated herein by reference in their entireties and to the same extent as if each reference was individually incorporated by reference.

It should be appreciated that as discussed herein, a subject may be a human or any animal. It should be appreciated that an animal may be a variety of any applicable type, including, but not limited thereto, mammal, veterinarian animal, livestock animal or pet type animal, etc. As an example, the animal may be a laboratory animal specifically selected to have certain characteristics similar to human (e.g. rat, dog, pig, monkey), etc. It should be appreciated that the subject may be any applicable human patient, for example.

As discussed herein, a “subject” may be any applicable human, animal, or other organism, living or dead, or other biological or molecular structure or chemical environment, and may relate to particular components of the subject, for instance specific tissues or fluids of a subject (e.g., human tissue in a particular area of the body of a living subject), which may be in a particular location of the subject, referred to herein as an “area of interest” or a “region of interest.”

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 10% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

### EXAMPLE

Practice of an aspect of an embodiment (or embodiments) of the invention will be still more fully understood from the following example and experimental results, which are presented herein for illustration only and should not be construed as limiting the invention in any way.

### Experimental Results

We conducted a retrospective analysis of 92 patients treated with Lung SBRT in the absence of chemotherapy. Kaplan-Meier curves, log-rank analysis and cox regression were performed to assess for survival differences associated with severe TRL and SPSS. We have developed a simulation for thorax RT to model circulating blood in treatment planning. In this, we considered radiation dose to circulating blood by coupling the time-dependence of the radiation delivery with a blood flow transport model that considers the transient time in regional structures as well as the mixing of irradiated and non-irradiated blood volumes.

### Modeling Lymphocyte Regeneration and Redistribution Rate

The model predicted the lymphocyte kill for each patient at 25 days since the beginning of treatment. Because the blood measurements were taken at different days for each patient, lymphocyte regeneration had to be applied to project the simulated, day 25, LYA values (blood cell sub type distribution) to day of measurement in order to compare the measurement to simulation. This was done by plotting the survival fraction, (1-K(D), the measurement of lymphocyte kill, which is also called lymph kill or cell kill, for each patient against the measurement day, and calculating the slope of the trend line. We assumed that patients with higher pre-treatment LYA would have healthier systems and a faster replenishment speed, and different rates were calculated for patients with different pre-treatment LYA values of (i) 0.5-1.0, (ii) 1.0-1.5, (iii) 1.5-2.0, (iv) >2.0 (cells/L)×10⁹. For a comparison of this method of different regeneration rates to one using an average rate across all patients, refer to Table II. Table II provides estimates of the difference in LYA (cells/L)×10⁹ after 100 days between the multi-stage regeneration and an average regeneration rate across all patients. The second column shows this difference for a patient in the middle of each LYA range if the measurement was taken after 100 days, and the third column gives the calculated regeneration rate.

TABLE II

| Pre Tx LYA | 100 Day Difference | LYA Regeneration Rate |
|---|---|---|
| (cells/L) × 10⁹ | (cells/L) × 10⁹ | (cells/L × 10⁹/day) |
| 0.5-1.0 | 0.14 | 0.0019 |
| 1.0-1.5 | 0.10 | 0.0011 |
| 1.5-2.0 | −0.04 | 0.0003 |
| 2.0-2.5 | −0.27 | −0.0004 |

### Multi-Fold Cross Validation Technique

The fractional lymphocyte kill function, also called the kill probability function, was optimized using a multi-fold cross-validation approach. The blood dose was calculated for each of 71 patients and separated into five groups. Each group was used four times in the training set and once in the test set. The optimized kill percentage at 0.5 Gy was then used to calculate K(Dᵢ) using the corresponding kill function used, and predict lymphocyte kill for each patient in the test set using the equation 1. The results were compared to measurements. Repeating this process with each patient subset taking a turn as the test set allowed for robust evaluation of the model accuracy while avoiding overfitting. All results reported in this paper are those calculated while the patient was part of the test set. The range of possible K(0.5Gy) values were used to obtain error bars on each prediction of post treatment LYA count.

### Validity of Model

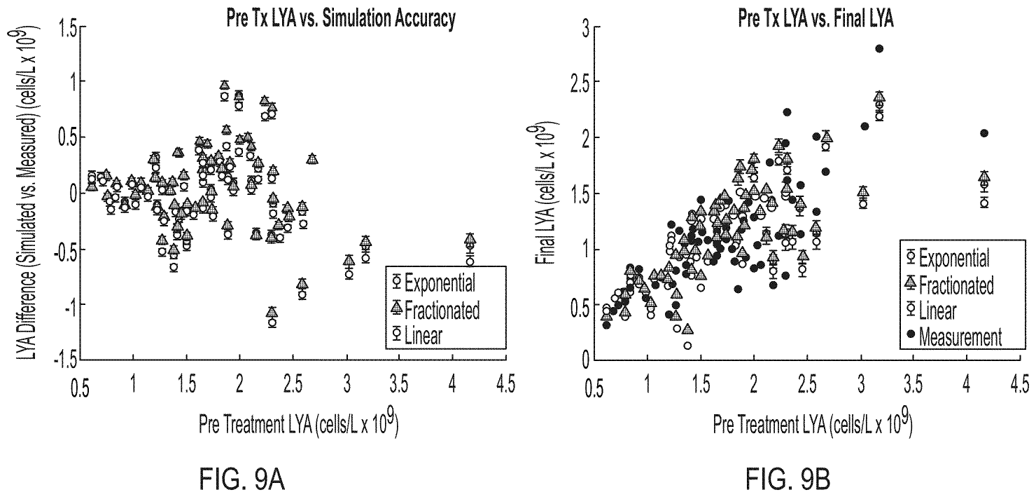

The new kill functions were used to calculate the final LYA value for each patient. Comparison of these values to measured values for each patient is given in FIGS. 9(A) and 9(B). FIG. 9(A) graphically demonstrates the simulation predicted and measured post treatment LYA count as a function of pre-treatment LYA count. FIG. 9(B) graphically demonstrates the LYA difference between simulation and measurement as a function of pre-treatment LYA value. The exponential kill function resulted in a predicted LYA decrease that differed from the measured values by an average of 0.31 (cells/L)×10⁹. For 40 of the 71 patients, the difference was less than 0.3 (cells/L)×10⁹. For the linear kill function, the average difference was 0.32 (cells/L)×10⁹, with 44 of the 71 patients showing a difference of less than 0.3 (cells/L)×10⁹. All learned kill models predicted to similar accuracy as the model given by Nakamura et al (27). Table III provides a summary of model accuracies. The first column gives the average LYA difference between measurement and results, and the second column gives the average percent difference. The “Min” and “Max” columns give the highest and lowest deviation between measurement and prediction. The next three columns show for how many patients each model was accurate to within 0.1, 0.3, and 0.5 (cells/L)×10⁹ final lymphocyte count respectively. The row labeled “Nakamura” shows the performance of the model predicted by Nakamura et al (27).

TABLE III

| | Absolute | | | | | | |
|---|---|---|---|---|---|---|---|
| | Difference | Percent | Min | Max | Num < | Num < | Num < |
| Kill Function | (cells/L × 10⁹) | Difference | (cells/L × 10⁹) | (cells/L × 10⁹) | 0.1 | 0.3 | 0.5 |
| Exponential | 0.30 (0.24) | 17 (12) | 0.0162 | 1.0921 | 14 | 40 | 61 |
| Linear | 0.29 (0.24) | 16 (12) | 0.0006 | 1.1671 | 13 | 44 | 60 |
| Fractionated | 0.29 (0.24) | 16 (11) | 0.0161 | 1.0651 | 17 | 43 | 62 |
| Nakamura | 0.29 (0.24) | 17 (12) | 0.0083 | 1.0277 | 15 | 42 | 60 |

Finally, this predictive model is able to predict the post-treatment absolute lymphocyte value to better than 16% across all variables of interest: age, pre-treatment

LYA value, post-treatment blood draw day, treatment delivery time, tumor volume size, and location of the tumor. Table IV provides a summary of accuracies of the predictive model within each subset of parameters. Parameters considered are: pre-treatment LYA count, age, day of post treatment blood draw, gated treatment, heart in the vicinity of treatment field, central vs peripheral, treatment delivery time, and ITV volume size.

TABLE IV

| | Absolute Difference | Percent | |
|---|---|---|---|
| | (cells/L × 10⁹) | Difference | Count |
| Pre Tx LYA | | | |
| <1.0 | 0.08 (0.04) | 11 (6) | 10 |
| 1.0-1.5 | 0.18 (0.14) | 14 (11) | 18 |
| 1.5-2.0 | 0.33 (0.24) | 19 (13) | 21 |
| >2.0 | 0.42 (0.27) | 18 (12) | 22 |
| Age | | | |
| <65 | 0.29 (0.21) | 16 (10) | 16 |
| 65-75 | 0.28 (0.22) | 17 (11) | 26 |
| 75-85 | 0.34 (0.30) | 17 (13) | 22 |
| >85 | 0.13 (0.09) | 7 (3) | 7 |
| Day | | | |
| <100 | 0.25 (0.21) | 15 (11) | 43 |
| 100-150 | 0.23 (0.17) | 12 (8) | 10 |
| 150-200 | 0.35 (0.38) | 18 (16) | 6 |
| >200 | 0.45 (0.28) | 20 (12) | 12 |
| Gated | | | |
| Yes | 0.28 (0.20) | 14 (8) | 15 |
| No | 0.29 (0.25) | 16 (12) | 55 |
| Heart near the PTV | | | |
| Yes | 0.28 (0.26) | 15 (11) | 40 |
| No | 0.29 (0.24) | 17 (12) | 26 |
| Tumor Location | | | |
| Central | 0.22 (0.16) | 14 (9) | 28 |
| Peripheral | 0.34 (0.28) | 17 (13) | 41 |
| Time | | | |
| <200 | 0.24 (0.21) | 15 (10) | 20 |
| 200-300 | 0.28 (0.23) | 15 (10) | 36 |
| >300 | 0.36 (0.30) | 20 (15) | 15 |
| Tumor Volume (cc) | | | |
| <20 | 0.40 (0.28) | 22 (13) | 17 |
| 20-40 | 0.25 (0.25) | 15 (10) | 24 |
| 40-60 | 0.25 (0.24) | 12 (10) | 12 |
| >60 | 0.24 (0.17) | 14 (9) | 17 |

### Sensitivity and Specificity of the Predictive Algorithm

Sensitivity and specificity of the predictive algorithm to detect a patient who will have close to grade three post treatment lymphopenia (LYA<0.8×10⁹ cells/L) was used to define the predictability of the algorithm. We chose to use grade LYA<0.8 as a threshold rather than a harsher but more widely known lymphopenic LYA level of 0.5×10⁹ cells/L because our dataset only had 5 patients with a final LYA count below this level.

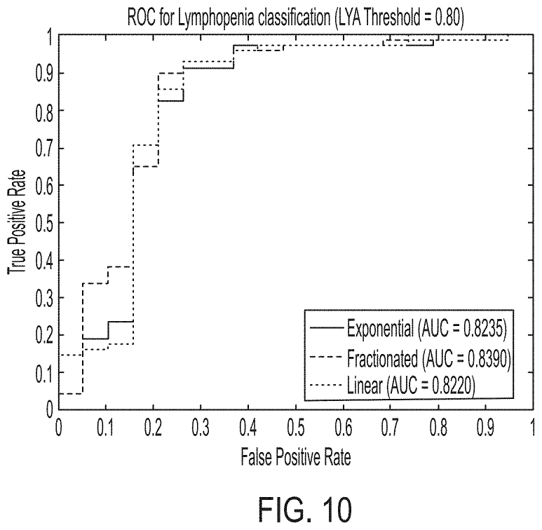

Receiver operating characteristic (ROC) analysis (35) was done in MATLAB for each learned kill function. The simulated LYA drop for each patient was used to predict whether that patient would develop post treatment lymphopenia. The patients which developed lymphopenia, as well as the simulated post-treatment LYA values for each patient, were used as arguments in the MATLAB perfcurve function to obtain the ROC information. The area under the curve (AUC) (36) of ROC was 0.84 for fractionated model, and 0.82 for exponential model. These results look very promising. FIG. 10 graphically demonstrates the ROC analysis results for a prediction of post treatment LYA count to be <0.8×10⁹ cells/L.

### Dose-Level Contributions to Lymphocyte Kill

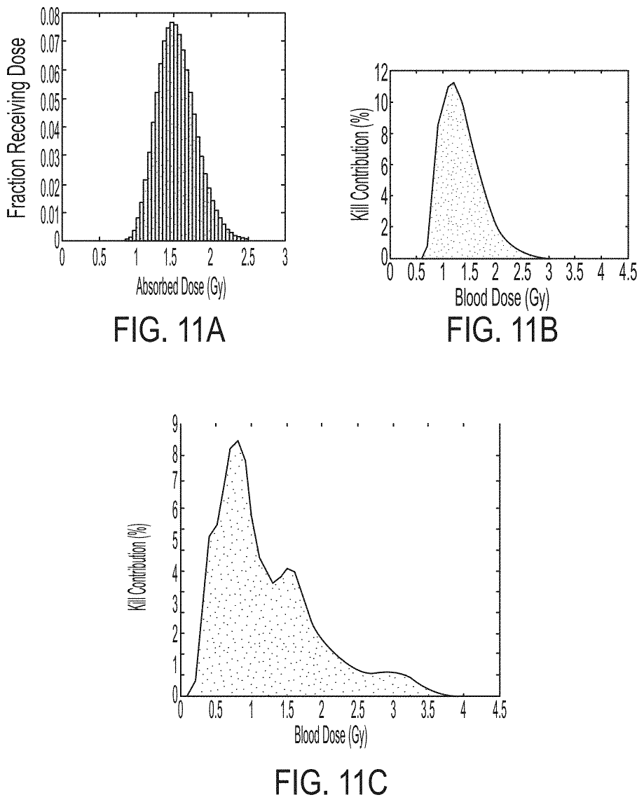

FIG. 11(A) graphically shows an example distribution of absorbed dose to blood cells from a single patient. FIG. 11(B) graphically shows the associated percentage kill contribution. In this case, the highest percentage of lymphocyte toxicity came from doses around 1.5 Gy. FIG. 11(C) graphically shows the kill contribution averaged across all patients. In general, lymphocyte toxicity was dominated by low dose levels (52% of toxicity came from doses <1 Gy) despite the lower cell kill probabilities at these levels. Only 14% of the lymphocyte toxicity came from cells absorbing more than 2 Gy.

### ADDITIONAL EXAMPLES

Example 1. A system for use in estimating the post-treatment blood cell sub type count of a subject treated via radiation therapy, said system comprising:

a computer processor;

a memory configured to store instructions that are executable by said computer processor, wherein said computer processor is configured to execute the instructions for:
performing processing associated with importing subject data into a simulation model;
performing processing associated with determining at least one time dependent dose for each voxel of at least one organ of said subject within said simulation model;
performing processing associated with creating a blood flow model for said at least one organ of said subject within said simulation model;
performing processing associated with simulating the delivery of a radiation dose to moving blood within said subject's body within said simulation model using said at least one time dependent dose for each voxel of said at least one organ of said subject and said blood flow model;
performing processing associated with determining at least one absorbed dose value for said subject's blood cell sub type within said simulation model;
performing processing associated with calculating a remaining blood cell sub type count; and
performing processing associated with transmitting said remaining blood cell sub type count to a secondary source.

Example 2. The system of example 1, wherein said secondary source includes one or more of anyone of the following:

local memory;

remote memory; or

display or graphical user interface.

Example 3. The system of example 1 (as well as subject matter in whole or in part of example 2), wherein said computer processor comprises at least one computer.

Example 4. The system of example 1 (as well as subject matter of one or more of any combination of examples 2-3, in whole or in part), wherein said system further comprises:

a server coupled to a network;

a user interface coupled to said network; and

an application coupled to said server and/or said user interface, wherein the application is configured for executing said computer processor.

Example 5. The system of example 1 (as well as subject matter of one or more of any combination of examples 2-4, in whole or in part), wherein said memory further comprises a main memory and a static memory.

Example 6. The system of example 1 (as well as subject matter of one or more of any combination of examples 2-5, in whole or in part), wherein said memory comprises one or more of anyone of the following:

electrically programmable read-only memory;

electrically erasable programmable read-only memory;

flash memory drive;

magnetic disk;

internal hard disk;

external hard disk;

removable disk;

magneto-optical disk;

CD-ROM disk; or

DVD-ROM disk.

Example 7. The system of example 1 (as well as subject matter of one or more of any combination of examples 2-6, in whole or in part), wherein said subject data includes any one or more of the following:

radiation therapy treatment plans;

molecular imaging planning image sets;

dose maps;

structure sets;

delivery times of said radiation dose;

blood cell sub type distribution

pre-treatment rate of regeneration;

pre-treatment rate of redistribution; or

subject age.

Example 8. The system of example 7, wherein said molecular imaging includes one of the following: computed tomography (CT), positron emission tomography (PET), ultrasound (US), magnetic resonance imaging (MRI), nuclear imaging, X-ray, single photon-emission computed tomography (SPECT), near-infrared tomography (NIRT), optical imaging, and optical computed tomography (OCT)

Example 9. The system of example 1 (as well as subject matter of one or more of any combination of examples 2-8, in whole or in part), wherein said simulation model is controlled by said computer processor.

Example 10. The system of example 1 (as well as subject matter of one or more of any combination of examples 2-9, in whole or in part), wherein said voxel is a three-dimensional shape within a three-dimensional matrix.

Example 11. The system of example 1 (as well as subject matter of one or more of any combination of examples 2-10, in whole or in part), wherein said blood cell sub type comprises lymphocytes.

Example 12. The system of example 11, wherein said lymphocytes includes any one or more of the following sub types:

CD3+;

CD4+;

CD8+;

CD19+; or

CD56+.

Example 13. The system of example 1 (as well as subject matter of one or more of any combination of examples 2-12, in whole or in part), wherein said at least one absorbed dose value is determined by a total blood volume, a heart-to-heart blood circulation time, a treatment delivery time, a dose delivered to moving blood, and said blood flow model.

Example 14. The at least one absorbed dose value of example 13, wherein said total blood volume is one of the following:

a range of about 2 to about 7 liters;

about 5 liters; or

a range of about 4 to about 6 liters.

Example 15. The at least one absorbed dose value of example 13 (as well as subject matter in whole or in part of example 14), wherein said heart-to-heart blood circulation time is one of the following:

a range of about 10 seconds to about 50 seconds;

about 30 seconds; or

a range of about 20 seconds to about 40 seconds.

Example 16. The at least one absorbed dose value of example 13, wherein said treatment delivery time is determined by a total delivered machine units and a dose rate of energy used.

Example 17. The at least one absorbed dose value of example 13 (as well as subject matter of one or more of any combination of examples 2-12 and 14-16, in whole or in part), wherein said dose delivered to moving blood is determined by:

dividing a total beam time into time steps;

applying said dose delivered to moving blood to a blood matrix;

rotating said blood matrix; and

randomly permuting blood.

Example 18. The system of example 1 (as well as subject matter in whole or in part of example 2-17), wherein said blood flow model includes organ specific cardiac outputs and blood velocities.

Example 19. The system of example 18, wherein said blood velocities vary from a center to at least one wall of great vessels.

Example 20. The system of example 1 (as well as subject matter in whole or in part of example 2-19), wherein said blood flow model comprises at least one logical mask, at least one dose map, at least one structure set, and at least one blood matrix.

Example 21. The blood flow model of example 20, wherein said at least one logical mask is provided by said at least one structure set.

Example 22. The blood flow model of example 21, wherein said at least one logical mask is applied for each organ.

Example 23. The blood flow model of example 22, wherein said at least one logical mask calculates a cross-sectional area in the z-direction.

Example 24. The blood flow model of example 23, wherein said cross-sectional area in the z-direction is used to shift said blood matrix.

Example 25. The blood flow model of example 24, wherein said blood matrix is shifted by the number of said voxels in said cross-sectional area of said at least one organ of said subject.

Example 26. The blood flow model of example 25, wherein an average blood density per voxel is determined for said at least one organ of said subject using the following formula:

$$v = \frac{\frac{\frac{5 Liters}{30 seconds} * CO * 1 layer}{cvoxels} * totalvoxels}{5 Liters}$$

wherein:

c is the number of voxels in one cross sectional layer,

CO is the cardiac output of the given organ, and

v is the result, wherein v is said average blood density per voxel.

Example 27. The blood flow model of example 26, further comprises wherein v is multiplied by a factor gv, wherein gv is a factor that accounts for higher blood density flowing through great vessels. .

Example 28. The blood flow model of example 27, wherein said blood matrix is rotated every one second per the average blood density per voxel.

Example 29. The system of example 1 (as well as subject matter in whole or in part of example 2-28), wherein said at least one time dependent dose is organ specific.

Example 30. The system of example 1 (as well as subject matter in whole or in part of example 2-29), wherein said remaining blood cell sub type count is determined by the following formula:

$$N ( t ) = {N}_{0} {\sum }_{i = 0}^{i = {N}_{0}} [ 1 - K ( {D}_{i} ) ] \text{/} {N}_{0} + R ( {N}_{0} - N ( t ) ) \cdot t \qquad ( 1 )$$

wherein:

Di is the absorbed dose values for the circulating blood/lymphocyte population;

K(D) is the kill probability function for a lymphocyte dependent on the dose (D) absorbed by the lymphocyte;

N(t), remaining blood cell sub type count, at a time t following radiation therapy is calculated by:

a time dependent net release rate of new lymphocytes to the circulating blood, defined as R(N0-N(t)); and wherein:
R(N0-N(t)) represents the combined effects of release from the lymphoid organs to blood, as well as a proliferation of the existing cells, and natural death of lymphocytes in blood.

Example 31. The system of example 30, wherein said time dependent net release rate of new lymphocytes to the circulating blood is configured to account for age and/or pre-treatment replenishment rates.

Example 32. The system of example 30 (as well as subject matter in whole or in part of example 31), wherein said kill probability function is determined by fitting at least one cell kill model to said subject data.

Example 33. The system of example 32, wherein said subject data includes any one or more of the following:

blood cell sub type distribution.

Example 34. The system of example 32 (as well as subject matter in whole or in part of example 2-33), wherein said at least one cell kill model is an exponential function using the linear-quadratic model determined by the following formula:

K(D)=1−exp(−αD−βD²).
wherein:
α and β are determined using the fixed condition that K(5Gy)=0.992; and
the value K(0.5Gy) is left free to vary.

Example 35. The system of example 32 (as well as subject matter of one or more of any combination of examples 2-31 and 33-24, in whole or in part), wherein said at least one cell kill model is a fractionated version of the linear quadratic model determined by the following formula:

K(D)=1−exp(−nd(α+βd))
wherein:
K(D) is the kill probability function for a lymphocyte dependent on the dose (D) absorbed by the lymphocyte;
α and β are determined using the fixed condition that K(5Gy)=0.992;
the value K(0.5Gy) is left free to vary; and
d is one fraction.

Example 36. The system of example 32 (as well as subject matter of one or more of any combination of examples 2-31 and 33-35, in whole or in part), wherein said at least one cell kill model is a point to point spline fit between each data point n=0 to 5, pₙ ∈ [0,0.5,2,3,4,5] determined by the following formula:

$$K ( D ) = \frac{K ( {p}_{n + 1} ) - K ( {p}_{n} )}{{p}_{n + 1} - {p}_{n}} ( D - {p}_{n} ) + K ( {p}_{n} )$$

wherein:
the data points are as follows:
   K(2Gy)=0.65;
   K(3Gy)=0.88;
   K(4Gy)=0.97; and
   K(5Gy)=0.992;

n is equal to the value of the first data point; and
a spline point for K(0.5Gy) is left free to vary.

Example 37. The system of example 32 (as well as subject matter of one or more of any combination of examples 2-31 and 33-36, in whole or in part), wherein said subject data further comprises a measured LYA reduction wherein the absolute value of said at least one cell kill model and said measured LYA reduction is decreased.

Example 38. The system of example 32 (as well as subject matter of one or more of any combination of examples 2-31 and 33-37, in whole or in part), wherein said kill probability function is graphically plotted against the measurement day of said subject to calculate the slope of the trend line.

Example 39. A computer method for estimating the post-treatment blood cell sub type count of a subject treated via radiation therapy, said method comprising:

performing processing associated with importing subject into a simulation model;

performing processing associated with determining at least one time dependent dose for each voxel of at least one organ of said subject within said simulation model;

performing processing associated with creating a blood flow model for said at least one organ of said subject within said simulation;

performing processing associated with simulating the delivery of a radiation dose to moving blood within said subject's body within said simulation model using said at least one time dependent dose for each voxel of said at least one organ of said subject and said blood flow model;

performing processing associated with determining at least one absorbed dose value for said subject's blood cell sub type within said simulation model;

performing processing associated with calculating a remaining blood cell sub type count; and

performing processing associated with transmitting said remaining blood cell sub type count to a secondary source.

Example 40. The method of example 39, wherein said secondary source includes one or more of anyone of the following:

local memory;

remote memory; or

display or graphical user interface.

Example 41. The method of example 39 (as well as subject matter in whole or in part of example 40), wherein said processing is accomplished by a computer processor or at least one computer.

Example 42. The method of example 39 (as well as subject matter of one or more of any combination of examples 40-41, in whole or in part), wherein said method further comprises:

communicating with a server coupled to a network;

performing processing associated with coupling a user interface to said network; and

performing processing associated with coupling an application to said server and/or said user interface, wherein the application is configured for performing processing.

Example 43. The method of example 40 (as well as subject matter of one or more of any combination of examples 41-42, in whole or in part), wherein said secondary source comprises a main memory and a static memory.

Example 44. The system of example 40 (as well as subject matter of one or more of any combination of examples 41-42, in whole or in part), wherein said secondary source comprises one or more of anyone of the following:

electrically programmable read-only memory;

electrically erasable programmable read-only memory;

flash memory drive;

magnetic disk;

internal hard disk;

external hard disk;

removable disk;

magneto-optical disk;

CD-ROM disk; or

DVD-ROM disk.

Example 45. The method of example 39 (as well as subject matter of one or more of any combination of examples 40-44, in whole or in part), wherein said subject data includes any one or more of the following:

radiation therapy treatment plans;

molecular imaging planning image sets;

dose maps;

structure sets;

delivery times of said radiation dose; or

blood cell sub type distribution;

pre-treatment rate of regeneration;

pre-treatment rate of redistribution; or

subject age.

Example 46. The method of example 45 (as well as subject matter of one or more of any combination of examples 40-44, in whole or in part), wherein said molecular imaging includes one of the following: computed tomography (CT), positron emission tomography (PET), ultrasound (US), magnetic resonance imaging (MRI), nuclear imaging, X-ray, single photon-emission computed tomography (SPECT), near-infrared tomography (NIRT), optical imaging, and optical computed tomography (OCT)

Example 47. The method of example 39 (as well as subject matter of one or more of any combination of examples 40-46, in whole or in part), wherein said simulation model is controlled by a computer processor.

Example 48. The method of example 39 (as well as subject matter of one or more of any combination of examples 40-47, in whole or in part), wherein said voxel is a three-dimensional shape within a three-dimensional matrix.

Example 49. The method of example 39 (as well as subject matter of one or more of any combination of examples 40-48, in whole or in part), wherein said blood cell sub type comprises lymphocytes.

Example 50. The method of example 49, wherein said lymphocytes includes any one or more of the following sub types:

CD3+;

CD4+;

CD8+;

CD19+; or

CD56+.

Example 51. The method of example 39 (as well as subject matter of one or more of any combination of examples 40-50, in whole or in part), wherein said at least one absorbed dose value is determined by a total blood volume, a heart-to-heart blood circulation time, a treatment delivery time, a dose delivered to moving blood, and said blood flow model.

Example 52. The at least one absorbed dose value of example 51, wherein said total blood volume is one of the following:

a range of about 2 to about 7 liters;

about 5 liters; or

a range of about 4 to about 6 liters.

Example 53. The at least one absorbed dose value of example 51 (as well as subject matter in whole or in part of example 52), wherein said heart-to-heart blood circulation time is one of the following:

a range of about 10 seconds to about 50 seconds;

about 30 seconds; or

a range of about 20 seconds to about 40 seconds.

Example 54. The at least one absorbed dose value of example 51 (as well as subject matter of one or more of any combination of examples 52-53, in whole or in part), wherein said treatment delivery time is determined by a total delivered machine units and a dose rate of energy used.

Example 55. The at least one absorbed dose value of example 51 (as well as subject matter of one or more of any combination of examples 52-54, in whole or in part), wherein said dose delivered to moving blood is determined by:

dividing a total beam time into time steps;

applying said dose to a blood matrix;

rotating said blood matrix; and

randomly permuting blood.

Example 56. The method of example 39 (as well as subject matter of one or more of any combination of examples 40-55, in whole or in part), wherein said blood flow model includes organ specific cardiac outputs and blood velocities.

Example 57. The method of example 56, wherein said blood velocities vary from a center to at least one wall of great vessels.

Example 58. The method of example 39 (as well as subject matter of one or more of any combination of examples 40-57, in whole or in part), wherein said blood flow model comprises at least one logical mask, at least one dose map, at least one structure set, and at least one blood matrix.

Example 59. The blood flow model of example 58, wherein said at least one logical mask is provided by said at least one structure set.

Example 60. The blood flow model of example 59, wherein said at least one logical mask is applied for each organ.

Example 61. The blood flow model of example 60, wherein said at least one logical mask calculates a cross-sectional area in the z-direction.

Example 62. The blood flow model of example 61, wherein said cross-sectional area in the z-direction is used to shift said blood matrix.

Example 63. The blood flow model of example 62, wherein said blood matrix is shifted by the number of said voxels in said cross-sectional area of said at least one organ of said subject.

Example 64. The blood flow model of example 63, wherein an average blood density per voxel is determined for said at least one organ of said subject using the following formula:

$$v = \frac{\frac{\frac{5 Liters}{30 seconds} * CO * 1 layer}{cvoxels} * totalvoxels}{5 Liters}$$

wherein:

c is the number of voxels in one cross sectional layer,

CO is the cardiac output of the given organ, and

v is the result, wherein v is said average blood density per voxel.

Example 65. The blood flow model of example 64, further comprises wherein v is multiplied by a factor gv, wherein gv is a factor that accounts for higher blood density flowing through great vessels.

Example 66. The blood flow model of example 65, wherein said blood matrix is rotated every one second per the average blood density per voxel.

Example 67. The method of example 39 (as well as subject matter of one or more of any combination of examples 40-66, in whole or in part), wherein said at least one time dependent dose is organ specific.

Example 68. The method of example 39 (as well as subject matter of one or more of any combination of examples 40-67, in whole or in part), wherein said remaining blood cell sub type count is determined by the following formula:

$$N ( t ) = {N}_{0} {\sum }_{i = 0}^{i = {N}_{0}} [ 1 - K ( {D}_{i} ) ] \text{/} {N}_{0} + R ( {N}_{0} - N ( t ) ) \cdot t \qquad ( 1 )$$

wherein:

Di is the absorbed dose values for the circulating blood/lymphocyte population;

K(D) is the kill probability function for a lymphocyte dependent on the dose (D) absorbed by the lymphocyte;

N(t), remaining blood cell sub type count, at a time t following radiation therapy is calculated by:

a time dependent net release rate of new lymphocytes to the circulating blood, defined as R(N0-N(t)); and wherein:

R(N0-N(t)) represents the combined effects of release from the lymphoid organs to blood, as well as a proliferation of the existing cells, and natural death of lymphocytes in blood.

Example 69. The method of example 68, wherein said time dependent net release rate of new lymphocytes to the circulating blood is configured to account for age and/or pre-treatment replenishment rates.

Example 70. The method of example 68 (as well as subject matter in whole or in part of example 69), wherein said kill probability function is determined by fitting at least one cell kill model to said subject data.

Example 71. The system of example 70, wherein said subject data includes any one or more of the following:

blood cell sub type distribution.

Example 72. The method of example 70 (as well as subject matter in whole or in part of example 71), wherein said at least one cell kill model is an exponential function using the linear-quadratic model determined by the following formula:

K(D)=1−exp(−αD−βD²).
wherein:
α and β are determined using the fixed condition that K(5Gy)=0.992; and
the value K(0.5Gy) is left free to vary.

Example 73. The method of example 70 (as well as subject matter of one or more of any combination of examples 71-72, in whole or in part), wherein said at least one cell kill model is a fractionated version of the linear quadratic model determined by the following formula:

K(D)=1−exp(−nd(α+βd))
wherein:
K(D) is the kill probability function for a lymphocyte dependent on the dose (D) absorbed by the lymphocyte;
α and β are determined using the fixed condition that K(5Gy)=0.992;
the value K(0.5Gy) is left free to vary; and
d is one fraction.

Example 74. The method of example 70 (as well as subject matter of one or more of any combination of examples 71-73, in whole or in part), wherein said at least one cell kill model is a point to point spline fit between each data point n=0 to 5, pₙ ∈ [0,0.5,2,3,4,5] determined by the following formula:

$$K ( D ) = \frac{K ( {p}_{n + 1} ) - K ( {p}_{n} )}{{p}_{n + 1} - {p}_{n}} ( D - {p}_{n} ) + K ( {p}_{n} )$$

wherein:
the data points are as follows:
   K(2Gy)=0.65;
   K(3Gy)=0.88;
   K(4Gy)=0.97; and
   K(5Gy)=0.992;
n is equal to the value of the first data point; and
a spline point for K(0.5Gy) is left free to vary.

Example 75. The method of example 70 (as well as subject matter of one or more of any combination of examples 71-74, in whole or in part), wherein said subject data further comprises a measured LYA reduction wherein the absolute value of said at least one cell kill model and said measured LYA reduction is decreased.

Example 76. The method of example 70 (as well as subject matter of one or more of any combination of examples 71-75, in whole or in part), wherein said kill probability function is graphically plotted against the measurement day of said subject to calculate the slope of the trend line.

Example 77. A non-transitory, computer readable storage medium having instructions stored thereon for use in estimating the post-treatment blood cell sub type count of a subject treated via radiation therapy that, when executed by a computer processor, cause the computer processor to:

receive subject data for a simulation model;

determine at least one time dependent dose for each voxel of at least one organ of said subject within said simulation model;

create a blood flow model for said at least one organ of said subject within said simulation model;

simulate the delivery of a radiation dose to moving blood within said subject's body using said at least one time dependent dose for each voxel of said at least one organ of said subject within said simulation model and said blood flow model;

determine at least one absorbed dose value for said subject's blood cell sub type;

calculate a remaining blood cell sub type count within said simulation model; and

transmit said remaining blood cell sub type count to a secondary source.

Example 78. The computer readable storage medium of example 77, wherein said secondary source includes one or more of anyone of the following:

local memory;

remote memory; or

display or graphical user interface.

Example 79. The computer readable storage medium of example 77 (as well as subject matter in whole or in part of example 78), wherein said computer processor comprises at least one computer.

Example 80. The computer readable storage medium of example 77 (as well as subject matter of one or more of any combination of examples 78-79, in whole or in part), wherein, when executed by the computer processor, causes the computer processor to communicate with:

a server coupled to a network;

a user interface coupled to said network; and

an application coupled to said server and/or said user interface, wherein the application is configured for executing said computer processor.

Example 81. The computer readable storage medium of example 78 (as well as subject matter of one or more of any combination of examples 79-80), wherein said secondary source comprises a main memory and a static memory.

Example 82. The computer readable storage medium of example 78 (as well as subject matter of one or more of any combination of examples 79-81), wherein said secondary source comprises one or more of anyone of the following:

electrically programmable read-only memory;

electrically erasable programmable read-only memory;

flash memory drive;

magnetic disk;

internal hard disk;

external hard disk;

removable disk;

magneto-optical disk;

CD-ROM disk; or

DVD-ROM disk.

Example 83. The computer readable storage medium of example 77 (as well as subject matter of one or more of any combination of examples 78-82), wherein said subject data includes any one or more of the following:

radiation therapy treatment plans;

molecular imaging planning image sets;

dose maps;

structure sets;

delivery times of said radiation dose; or

blood cell sub type distribution;

pre-treatment rate of regeneration;

pre-treatment rate of redistribution; or

subject age.

Example 84. The computer readable storage medium of example 83, wherein said molecular imaging includes one of the following: computed tomography (CT), positron emission tomography (PET), ultrasound (US), magnetic resonance imaging (MRI), nuclear imaging, X-ray, single photon-emission computed tomography (SPECT), near-infrared tomography (NIRT), optical imaging, and optical computed tomography (OCT)

Example 85. The computer readable storage medium of example 77 (as well as subject matter of one or more of any combination of examples 78-84), wherein said simulation model is controlled by said computer processor.

Example 86. The computer readable storage medium of example 77 (as well as subject matter of one or more of any combination of examples 78-85), wherein said voxel is a three-dimensional shape within a three-dimensional matrix.

Example 87. The computer readable storage medium of example 77 (as well as subject matter of one or more of any combination of examples 78-86), wherein said blood cell sub type comprises lymphocytes.

Example 88. The computer readable storage medium of example 87, wherein said lymphocytes includes any one or more of the following sub types:

CD3+;

CD4+;

CD8+;

CD19+; or

CD56+.

Example 89. The computer readable storage medium of example 77 (as well as subject matter of one or more of any combination of examples 78-88), wherein said at least one absorbed dose value is determined by a total blood volume, a heart-to-heart blood circulation time, a treatment delivery time, a dose delivered to moving blood, and said blood flow model.

Example 90. The at least one absorbed dose value of example 89, wherein said total blood volume is one of the following:

a range of about 2 to about 7 liters;

about 5 liters; or

a range of about 4 to about 6 liters.

Example 91. The at least one absorbed dose value of example 89 (as well as subject matter in whole or in part of example 90), wherein said heart-to-heart blood circulation time is

one of the following:

a range of about 10 seconds to about 50 seconds;

about 30 seconds; or

a range of about 20 seconds to about 40 seconds.

Example 92. The at least one absorbed dose value of example 89 (as well as subject matter of one or more of any combination of examples 90-91), wherein said treatment delivery time is determined by a total delivered machine units and a dose rate of energy used.

Example 93. The at least one absorbed dose value of example 89 (as well as subject matter of one or more of any combination of examples 90-92), wherein said dose delivered to moving blood is determined by:

dividing a total beam time into time steps;

applying said dose to a blood matrix;

rotating said blood matrix; and

randomly permuting blood.

Example 94. The computer readable storage medium of example 77 (as well as subject matter of one or more of any combination of examples 78-93), wherein said blood flow model includes organ specific cardiac outputs and blood velocities.

Example 95. The computer readable storage medium of example 94, wherein said blood velocities vary from a center to at least one wall of great vessels.

Example 96. The computer readable storage medium of example 77 (as well as subject matter of one or more of any combination of examples 78-95), wherein said blood flow model comprises at least one logical mask, at least one dose map, at least one structure set, and at least one blood matrix.

Example 97. The blood flow model of example 96, wherein said at least one logical mask is provided by said at least one structure set.

Example 98. The blood flow model of example 97, wherein said at least one logical mask is applied for each organ.

Example 99. The blood flow model of example 98, wherein said at least one logical mask calculates a cross-sectional area in the z-direction.

Example 100. The blood flow model of example 99, wherein said cross-sectional area in the z-direction is used to shift said blood matrix.

Example 101. The blood flow model of example 100, wherein said blood matrix is shifted by the number of said voxels in said cross-sectional area of said at least one organ of said subject.

Example 102. The blood flow model of example 101, wherein an average blood density per voxel is determined for said at least one organ of said subject using the following formula:

$$v = \frac{\frac{\frac{5 Liters}{30 seconds} * CO * 1 layer}{cvoxels} * totalvoxels}{5 Liters}$$

wherein:

c is the number of voxels in one cross sectional layer,

CO is the cardiac output of the given organ, and

v is the result, wherein v is said average blood density per voxel.

Example 103. The blood flow model of example 102, further comprises wherein v is multiplied by a factor gv, wherein gv is a factor that accounts for higher blood density flowing through great vessels.

Example 104. The blood flow model of example 103, wherein said blood matrix is rotated every one second per the average blood density per voxel.

Example 105. The computer readable storage medium of example 77 (as well as subject matter of one or more of any combination of examples 78-104), wherein said at least one time dependent dose is organ specific.

Example 106. The computer readable storage medium of example 77 (as well as subject matter of one or more of any combination of examples 78-105), wherein said remaining blood cell sub type count is determined by the following formula:

$$N ( t ) = {N}_{0} {\sum }_{i = 0}^{i = {N}_{0}} [ 1 - K ( {D}_{i} ) ] \text{/} {N}_{0} + R ( {N}_{0} - N ( t ) ) \cdot t \qquad ( 1 )$$

wherein:

Di is the absorbed dose values for the circulating blood/lymphocyte population;

K(D) is the kill probability function for a lymphocyte dependent on the dose (D) absorbed by the lymphocyte;

N(t), remaining blood cell sub type count, at a time t following radiation therapy is calculated by:

a time dependent net release rate of new lymphocytes to the circulating blood, defined as R(N0-N(t)); and wherein:

R(N0-N(t)) represents the combined effects of release from the lymphoid organs to blood, as well as a proliferation of the existing cells, and natural death of lymphocytes in blood.

Example 107. The computer readable storage medium of example 106, wherein said time dependent net release rate of new lymphocytes to the circulating blood is configured to account for age and/or pre-treatment replenishment rates.

Example 108. The computer readable storage medium of example 106 (as well as subject matter in whole or in part of example 107), wherein said kill probability function is determined by fitting at least one cell kill model to said subject data.

Example 109. The computer readable storage medium of example 108, wherein said subject data includes any one or more of the following:

blood cell sub type distribution.

Example 110. The computer readable storage medium of example 108 (as well as subject matter in whole or in part of example 109), wherein said at least one cell kill model is an exponential function using the linear-quadratic model determined by the following formula:

K(D)=1−exp(−αD−βD²).
wherein:
α and β are determined using the fixed condition that K(5Gy)=0.992; and
the value K(0.5Gy) is left free to vary.

Example 111. The computer readable storage medium of example 108 (as well as subject matter of one or more of any combination of examples 109-110), wherein said at least one cell kill model is a fractionated version of the linear quadratic model determined by the following formula:

K(D)=1−exp(−nd(α+βd))
wherein:
K(D) is the kill probability function for a lymphocyte dependent on the dose (D) absorbed by the lymphocyte;
α and β are determined using the fixed condition that K(5Gy)=0.992;
the value K(0.5Gy) is left free to vary; and
d is one fraction.

Example 112. The computer readable storage medium of example 108 (as well as subject matter of one or more of any combination of examples 109-111), wherein said at least one cell kill model is a point to point spline fit between each data point n=0 to 5, pₙ ∈ [0,0.5,2,3,4,5] determined by the following formula:

$$K ( D ) = \frac{K ( {p}_{n + 1} ) - K ( {p}_{n} )}{{p}_{n + 1} - {p}_{n}} ( D - {p}_{n} ) + K ( {p}_{n} )$$

wherein:
the data points are as follows:
   K(2Gy)=0.65;
   K(3Gy)=0.88;
   K(4Gy)=0.97; and
   K(5Gy)=0.992;

n is equal to the value of the first data point; and
a spline point for K(0.5Gy) is left free to vary.

Example 113. The computer readable storage medium of example 108 (as well as subject matter of one or more of any combination of examples 109-112), wherein said subject data further comprises a measured LYA reduction wherein the absolute value of said at least one cell kill model and said measured LYA reduction is decreased.

Example 114. The computer readable storage medium of example 108 (as well as subject matter of one or more of any combination of examples 109-113), wherein said kill probability function is graphically plotted against the measurement day of said subject to calculate the slope of the trend line.

Example 115. A system configured to perform the method of any one or more of Examples 39-76.

Example 116. A computer program product configured to perform the method of any one or more of Examples 39-76.

Example 117. The method of using any of the elements, components, devices, computer program product and/or systems or their sub-components provided in any one or more of examples 1-114, in whole or in part.

Example 118. The method of manufacturing any of the elements, components, devices, computer program product, and/or systems or their sub-components provided in any one or more of examples 1-114, in whole or in part.

### REFERENCES

The devices, systems, apparatuses, modules, compositions, materials, computer program products, non-transitory computer readable medium, and methods of various embodiments of the invention disclosed herein may utilize aspects (such as devices, apparatuses, modules, systems, compositions, materials, computer program products, non-transitory computer readable medium, and methods) disclosed in the following references, applications, publications and patents and which are hereby incorporated by reference herein in their entirety (and which are not admitted to be prior art with respect to the present invention by inclusion in this section).

1. Shohan J. Some theoretical considerations on the present status of roentgen therapy. N Engl J Med. 1916; 175:321-327.

2. Maclennan I C M, Kay H E M. Analysis of treatment in childhood leukemia. IV. The critical association between dose fractionation and immunosuppression induced by cranial irradiation. Cancer. 1978; 41(1):108-11.

3. Weeke E. The development of lymphopenia in uremic patients undergoing extracorporeal irradiation of the blood with portable beta units. Radiat Res. 1973; 56(3):554-9.

4. Grossman S A, Ye X, Lesser G, Sloan A, Carraway H, Desideri S, et al. Immunosuppression in patients with high-grade gliomas treated with radiation and temozolomide. Clin Cancer Res. 2011; 17(16):5473-80.

5. Campian J L, Ye X, Brock M, Grossman S A. Treatment-related lymphopenia in patients with stage III non-small-cell lung cancer. Cancer Invest. 2013; 31(3):183-8.

6. Balmanoukian A, Ye X, Herman J, Laheru D, Grossman S A. The association between treatment-related lymphopenia and survival in newly diagnosed patients with resected adenocarcinoma of the pancreas. Cancer Invest. 2012; 30(8):571-6.

7. Wild A T, Ye X, Ellsworth S G, Smith J A, Narang A K, Garg T, et al. The Association Between Chemoradiation-related Lymphopenia and Clinical Outcomes in Patients With Locally Advanced Pancreatic Adenocarcinoma. Am J Clin Oncol. 2015; 38(3):259-65.

8. Campian J L, Sarai G, Ye X, Marur S, Grossman S A. Association between severe treatment-related lymphopenia and progression-free survival in patients with newly diagnosed squamous cell head and neck cancer. Head Neck. 2014; 36(12):1747-53.

9. Grossman S A, Ellsworth S, Campian J, Wild A T, Herman J M, Laheru D, et al. Survival in Patients With Severe Lymphopenia Following Treatment With Radiation and Chemotherapy for Newly Diagnosed Solid Tumors. J Natl Compr Canc Netw. 2015; 13(10):1225-31.

10. Hughes M A, Parisi M, Grossman S, Kleinberg L. Primary brain tumors treated with steroids and radiotherapy: Low CD4 counts and risk of infection. Int J Radiat Oncol Biol Phys. 2005; 62(5):1423-6.

11. Kaplan E L, Meier P. Nonparametric Estimation from Incomplete Observations. J Am Stat Assoc. 1958;

12. Wild A T, Herman J M, Dholakia A S, Moningi S, Lu Y, Rosati L M, et al. Lymphocyte-Sparing Effect of Stereotactic Body Radiation Therapy in Patients with Unresectable Pancreatic Cancer. Int J Radiat Oncol Biol Phys. 2016; 94(3):571-9.

13. Zhao X, Yan X, Ju Y F, Yu H M, Jiao S C. [Expression and clinical significance of CD3, CD4 and COX-2 in non-small cell lung cancer]. Xi Bao Yu Fen Zi Mian Yi Xue Za Zhi. 2012; 28(4):407-9.

14. Pittet M J. Behavior of immune players in the tumor microenvironment. Vol. 21, Current Opinion in Oncology. 2009. p. 53-9.

15. Greenhough A, Smartt H J M, Moore A E, Roberts H R, Williams A C, Paraskeva C, et al. The COX-2/PGE2 pathway: Key roles in the hallmarks of cancer and adaptation to the tumour microenvironment. Carcinogenesis. 2009; 30(3):377-86.

16. Yu Q M, Yu C D, Ling Z Q. Elevated circulating CD19+ lymphocytes predict survival advantage in patients with gastric cancer. Asian Pac J Cancer Prey. 2012; 13(5):2219-24.

17. Kim H-J, Cantor H. CD4 T-cell Subsets and Tumor Immunity: The Helpful and the Not-so-Helpful. Cancer Immunol Res. 2014; 2(2):91-8.

18. Protti M P, Monte L D, Lullo G D. Tumor antigen-specific CD4+ T cells in cancer immunity: From antigen identification to tumor prognosis and development of therapeutic strategies. Vol. 83, Tissue Antigens. 2014. p. 237-46.

19. Zhang L, Romero P. Metabolic Control of CD8+ T Cell Fate Decisions and Antitumor Immunity. Trends Mol Med. 2017;

20. Scheuermann R H, Racila E. CD19 antigen in leukemia and lymphoma diagnosis and immunotherapy. Leuk Lymphoma. 1995; 18(5-6):385-97.

21. Wagner J A, Rosario M, Romee R, Berrien-Elliott M M, Schneider S E, Leong J W, et al. CD56brightNK cells exhibit potent antitumor responses following IL-15 priming. J Clin Invest. 2017; 127(11).

22. Poli A, Michel T, Thérésine M, Andrès E, Hentges F, Zimmer J. CD56bright natural killer (NK) cells: An important NK cell subset. Vol. 126, Immunology. 2009. p. 458-65.

23. Yovino S, Kleinberg L, Grossman S A, Narayanan M, Ford E. The etiology of treatment-related lymphopenia in patients with malignant gliomas: Modeling radiation dose to circulating lymphocytes explains clinical observations and suggests methods of modifying the impact of radiation on immune cells. Cancer Invest. 2013; 31(2):140-4.

24. Leggett R W, Williams L R. A proposed blood circulation model for reference man. Health Phys. 1995; 69(2):187-201.

25. Williams L R, Leggett R W. Reference values for resting blood flow to organs of man. Vol. 10, Clinical Physics and Physiological Measurement. 1989. p. 187-217.

26. Gabe I T, Gault J H, Ross J R, J, Mason, D T, Mills C J, Schillingford J P, and Braunwald E., Measurement of Instantaneous Blood Flow Velocity and Pressure in Conscious Man with a Catheter-Tip Velocity Probe; Journal of the American heart association, Vol XL (5), 1969.

27. Nakamura N, Kusunoki Y, Akiyama M. Radiosensitivity of CD4 or CD8 positive human T-lymphocytes by an in vitro colony formation assay. Radiat Res [Internet]. 1990; 123(2):224-7. Available from: http://www.ncbi.nlm.nih.gov/pubmed/2117766

28. L. Basler, N. Andratschke, S. Ehrbar M G and ST-L. Modelling the immunosuppressive effect of liver SBRT by simulating the dose to circulating lymphocytes: an in-silico planning study. Radiat Oncol. 2018; 1-8.

29. Douglas B G, Fowler J F. The Effect of Multiple Small Doses of X Rays on Skin Reactions in the Mouse and a Basic Interpretation. Radiat Res. 2012;

30. Brenner D J. The Linear-Quadratic Model Is an Appropriate Methodology for Determining Isoeffective Doses at Large Doses Per Fraction. Semin Radiat Oncol. 2008;

31. McMullen T., Liyanage S., Alonso C., Wijesooriya K. Radiation Related Lymphopenia for Lung SBRT Treatments: Measurement Versus Simulation. Med Phys. 2017; 44(6):3118.

32. Fülöp T, Larbi A, Pawelec G. Human T cell aging and the impact of persistent viral infections. Vol. 4, Frontiers in Immunology. 2013.

33. Larbi A, Rymkiewicz P, Vasudev A, Low I, Shadan N B, Mustafah S, et al. The immune system in the elderly: a fair fight against diseases? Vol. 9, Aging Health. 2013. p. 35-47.

34. Le Saux S, Weyand C M, Goronzy J J. Mechanisms of immunosenescence: Lessons from models of accelerated immune aging. Ann N Y Acad Sci. 2012; 1247(1):69-82.

35. McNeil B J, Hanley J A. Statistical approaches to the analysis of receiver operating characteristic (ROC) curves. Med Decis Making. 1984;

36. Fawcett T. An introduction to ROC analysis. Pattern Recognit Lett. 2006;

37. U.S. Pat. No. 10,105,555 B2, Ellsworth, et al., “System and Method for Determining Radiation Dose to Circulating Blood”, Oct. 23, 2018.

38. U.S. Patent Application Publication No. US 2016/0135901 A1, Raghavan, et al., “Treatment Simulator for Brain Diseases and Method of Use Thereof”, May 19, 2016.

39. U.S. Patent Application Publication No. US 2016/0287903 A1, Sgouros, et al., “System and Method for Determining Amount of Radioactive Material to Administer to a Patient”, Oct. 6, 2016.

40. U.S. Pat. No. 8,693,629 B2, Sgouros, et al., “Method and System for Administering Internal Radionuclide Therapy (IRT) and External Radiation Therapy (XRT), Apr. 8, 2014.

41. International Patent Application Publication No. WO 2014/066798 A2, Hobbs, et al., “Method and System for Treating a Disease Using Combined Radiopharmaceuticals”, May 1, 2014.

42. U.S. Patent Application Publication No. US 2013/0165732 A1, Sgouros, et al., “Method and System for Administering Radiopharmaceutical Therapy (RPT)”, Jun. 27, 2013.

43. U.S. Patent Application Publication No. US 2015/0286796 A1, Hobbs, et al., “Method and System for Treating a Disease Using Combined Radiopharmaceuticals”, Oct. 8, 2015.

44. U.S. Pat. No. 9,265,577 B2, Raghavan, et al., “Methods and Systems for Providing Planning and Dispensation of Research and/or Treatment for Brain Disease”, Feb. 23, 2016.

45. U.S. Patent Application Publication No. US 2016/0310018 A1, Fonte, et al., “Systems and Methods for Estimating Ischemia and Blood Flow Characteristics from Vessel Geometry and Physiology”, Oct. 27, 2016.

In summary, while the present invention has been described with respect to specific embodiments, many modifications, variations, alterations, substitutions, and equivalents will be apparent to those skilled in the art. The present invention is not to be limited in scope by the specific embodiment described herein. Indeed, various modifications of the present invention, in addition to those described herein, will be apparent to those of skill in the art from the foregoing description and accompanying drawings. Accordingly, the invention is to be considered as limited only by the spirit and scope of the disclosure (and claims), including all modifications and equivalents.

Still other embodiments will become readily apparent to those skilled in this art from reading the above-recited detailed description and drawings of certain exemplary embodiments. It should be understood that numerous variations, modifications, and additional embodiments are possible, and accordingly, all such variations, modifications, and embodiments are to be regarded as being within the spirit and scope of this application. For example, regardless of the content of any portion (e.g., title, field, background, summary, abstract, drawing figure, etc.) of this application, unless clearly specified to the contrary, there is no requirement for the inclusion in any claim herein or of any application claiming priority hereto of any particular described or illustrated activity or element, any particular sequence of such activities, or any particular interrelationship of such elements. Moreover, any activity can be repeated, any activity can be performed by multiple entities, and/or any element can be duplicated. Further, any activity or element can be excluded, the sequence of activities can vary, and/or the interrelationship of elements can vary. Unless clearly specified to the contrary, there is no requirement for any particular described or illustrated activity or element, any particular sequence or such activities, any particular size, speed, material, dimension or frequency, or any particularly interrelationship of such elements. Accordingly, the descriptions and drawings are to be regarded as illustrative in nature, and not as restrictive. Moreover, when any number or range is described herein, unless clearly stated otherwise, that number or range is approximate. When any range is described herein, unless clearly stated otherwise, that range includes all values therein and all sub ranges therein. Any information in any material (e.g., a United States/foreign patent, United States/foreign patent application, book, article, etc.) that has been incorporated by reference herein, is only incorporated by reference to the extent that no conflict exists between such information and the other statements and drawings set forth herein. In the event of such conflict, including a conflict that would render invalid any claim herein or seeking priority hereto, then any such conflicting information in such incorporated by reference material is specifically not incorporated by reference herein.

What is claimed is:

1. A system for use in estimating the post-treatment blood cell sub type count of a subject treated via radiation therapy, said system comprising:
a computer processor;
a memory configured to store instructions that are executable by said computer processor, wherein said computer processor is configured to execute the instructions for:
performing processing associated with importing subject data into a simulation model;
performing processing associated with determining at least one time dependent dose for each voxel of at least one organ of said subject within said simulation model;
performing processing associated with creating a blood flow model for said at least one organ of said subject within said simulation model;
performing processing associated with simulating the delivery of a radiation dose to moving blood within said subject's body within said simulation model using said at least one time dependent dose for each voxel of said at least one organ of said subject and said blood flow model;
performing processing associated with determining at least one absorbed dose value for said subject's blood cell sub type within said simulation model;
performing processing associated with calculating a remaining blood cell sub type count; and
performing processing associated with transmitting said remaining blood cell sub type count to a secondary source.

2. The system of claim 1, wherein said secondary source includes one or more of anyone of the following:
local memory;
remote memory; or
display or graphical user interface.

3. The system of claim 1, wherein said computer processor comprises at least one computer.

4. The system of claim 1, wherein said system further comprises:
a server coupled to a network;
a user interface coupled to said network; and
an application coupled to said server and/or said user interface, wherein the application is configured for executing said computer processor.

5. The system of claim 1, wherein said memory further comprises a main memory and a static memory.

6. The system of claim 1, wherein said memory comprises one or more of anyone of the following:
electrically programmable read-only memory;
electrically erasable programmable read-only memory;
flash memory drive;
magnetic disk;
internal hard disk;
external hard disk;
removable disk;
magneto-optical disk;
CD-ROM disk; or
DVD-ROM disk.

7. The system of claim 1, wherein said subject data includes any one or more of the following:
radiation therapy treatment plans;
molecular imaging planning image sets;
dose maps;
structure sets;
delivery times of said radiation dose;
blood cell sub type distribution
pre-treatment rate of regeneration;
pre-treatment rate of redistribution; or
subject age.

8. The system of claim 7, wherein said molecular imaging includes one of the following: computed tomography (CT), positron emission tomography (PET), ultrasound (US), magnetic resonance imaging (MRI), nuclear imaging, X-ray, single photon-emission computed tomography (SPECT), near-infrared tomography (NIRT), optical imaging, and optical computed tomography (OCT)

9. The system of claim 1, wherein said simulation model is controlled by said computer processor.

10. The system of claim 1, wherein said voxel is a three-dimensional shape within a three-dimensional matrix.

11. The system of claim 1, wherein said blood cell sub type comprises lymphocytes.

12. The system of claim 11, wherein said lymphocytes includes any one or more of the following sub types:
CD3+;
CD4+;
CD8+;
CD19+; or
CD56+.

13. The system of claim 1, wherein said at least one absorbed dose value is determined by a total blood volume, a heart-to-heart blood circulation time, a treatment delivery time, a dose delivered to moving blood, and said blood flow model.

14. The at least one absorbed dose value of claim 13, wherein said total blood volume is one of the following:
a range of about 2 to about 7 liters;
about 5 liters; or
a range of about 4 to about 6 liters.

15. The at least one absorbed dose value of claim 13, wherein said heart-to-heart blood circulation time is one of the following:
a range of about 10 seconds to about 50 seconds;
about 30 seconds; or
a range of about 20 seconds to about 40 seconds.

16. The at least one absorbed dose value of claim 13, wherein said treatment delivery time is determined by a total delivered machine units and a dose rate of energy used.

17. The at least one absorbed dose value of claim 13, wherein said dose delivered to moving blood is determined by:
dividing a total beam time into time steps;
applying said dose delivered to moving blood to a blood matrix;
rotating said blood matrix; and
randomly permuting blood.

18. The system of claim 1, wherein said blood flow model includes organ specific cardiac outputs and blood velocities.

19. The system of claim 18, wherein said blood velocities vary from a center to at least one wall of great vessels.

20. The system of claim 1, wherein said blood flow model comprises at least one logical mask, at least one dose map, at least one structure set, and at least one blood matrix.

21. The blood flow model of claim 20, wherein said at least one logical mask is provided by said at least one structure set.

22. The blood flow model of claim 21, wherein said at least one logical mask is applied for each organ.

23. The blood flow model of claim 22, wherein said at least one logical mask calculates a cross-sectional area in the z-direction.

24. The blood flow model of claim 23, wherein said cross-sectional area in the z-direction is used to shift said blood matrix.

25. The blood flow model of claim 24, wherein said blood matrix is shifted by the number of said voxels in said cross-sectional area of said at least one organ of said subject.

26. The blood flow model of claim 25, wherein an average blood density per voxel is determined for said at least one organ of said subject using the following formula:
$$v = \frac{\frac{\frac{5 Liters}{30 seconds} * CO * 1 layer}{cvoxels} * totalvoxels}{5 Liters}$$
wherein:
c is the number of voxels in one cross sectional layer,
CO is the cardiac output of the given organ, and
v is the result, wherein v is said average blood density per voxel.

27. The blood flow model of claim 26, further comprises wherein v is multiplied by a factor gv, wherein gv is a factor that accounts for higher blood density flowing through great vessels. .

28. The blood flow model of claim 27, wherein said blood matrix is rotated every one second per the average blood density per voxel.

29. The system of claim 1, wherein said at least one time dependent dose is organ specific.

30. The system of claim 1, wherein said remaining blood cell sub type count is determined by the following formula:
$$N ( t ) = {N}_{0} {\sum }_{i = 0}^{i = {N}_{0}} [ 1 - K ( {D}_{i} ) ] \text{/} {N}_{0} + R ( {N}_{0} - N ( t ) ) \cdot t \qquad ( 1 )$$
wherein:
Di is the absorbed dose values for the circulating blood/lymphocyte population;
K(D) is the kill probability function for a lymphocyte dependent on the dose (D) absorbed by the lymphocyte;
N(t), remaining blood cell sub type count, at a time t following radiation therapy is calculated by:
a time dependent net release rate of new lymphocytes to the circulating blood, defined as R(N0-N(t)); and wherein:
R(N0-N(t)) represents the combined effects of release from the lymphoid organs to blood, as well as a proliferation of the existing cells, and natural death of lymphocytes in blood.

31. The system of claim 30, wherein said time dependent net release rate of new lymphocytes to the circulating blood is configured to account for age and/or pre-treatment replenishment rates.

32. The system of claim 30, wherein said kill probability function is determined by fitting at least one cell kill model to said subject data.

33. The system of claim 32, wherein said subject data includes any one or more of the following:
blood cell sub type distribution.

34. The system of claim 32, wherein said at least one cell kill model is an exponential function using the linear-quadratic model determined by the following formula:
K(D)=1−exp(−αD−βD²).
wherein:
α and β are determined using the fixed condition that K(5Gy)=0.992; and
the value K(0.5Gy) is left free to vary.

35. The system of claim 32, wherein said at least one cell kill model is a fractionated version of the linear quadratic model determined by the following formula:
K(D)=1−exp(−nd(α+βd))
wherein:
K(D) is the kill probability function for a lymphocyte dependent on the dose (D) absorbed by the lymphocyte;
α and β are determined using the fixed condition that K(5Gy)=0.992;
the value K(0.5Gy) is left free to vary; and
d is one fraction.

36. The system of claim 32, wherein said at least one cell kill model is a point to point spline fit between each data point n=0 to 5, pₙ ∈ [0,0.5,2,3,4,5] determined by the following formula:
$$K ( D ) = \frac{K ( {p}_{n + 1} ) - K ( {p}_{n} )}{{p}_{n + 1} - {p}_{n}} ( D - {p}_{n} ) + K ( {p}_{n} )$$
wherein:
the data points are as follows:
K(2Gy)=0.65;
K(3Gy)=0.88;
K(4Gy)=0.97; and
K(5Gy)=0.992;
n is equal to the value of the first data point; and
a spline point for K(0.5Gy) is left free to vary.

37. The system of claim 32, wherein said subject data further comprises a measured LYA reduction wherein the absolute value of said at least one cell kill model and said measured LYA reduction is decreased.

38. The system of claim 32, wherein said kill probability function is graphically plotted against the measurement day of said subject to calculate the slope of the trend line.

39. A computer method for estimating the post-treatment blood cell sub type count of a subject treated via radiation therapy, said method comprising:
performing processing associated with importing subject into a simulation model;
performing processing associated with determining at least one time dependent dose for each voxel of at least one organ of said subject within said simulation model;
performing processing associated with creating a blood flow model for said at least one organ of said subject within said simulation;
performing processing associated with simulating the delivery of a radiation dose to moving blood within said subject's body within said simulation model using said at least one time dependent dose for each voxel of said at least one organ of said subject and said blood flow model;
performing processing associated with determining at least one absorbed dose value for said subject's blood cell sub type within said simulation model;
performing processing associated with calculating a remaining blood cell sub type count; and
performing processing associated with transmitting said remaining blood cell sub type count to a secondary source.

40. The method of claim 39, wherein said secondary source includes one or more of anyone of the following:
local memory;
remote memory; or
display or graphical user interface.

41. The method of claim 39, wherein said processing is accomplished by a computer processor or at least one computer.

42. The method of claim 39, wherein said method further comprises:
communicating with a server coupled to a network;
performing processing associated with coupling a user interface to said network; and
performing processing associated with coupling an application to said server and/or said user interface, wherein the application is configured for performing processing.

43. The method of claim 40, wherein said secondary source comprises a main memory and a static memory.

44. The system of claim 40, wherein said secondary source comprises one or more of anyone of the following:
electrically programmable read-only memory;
electrically erasable programmable read-only memory;
flash memory drive;
magnetic disk;
internal hard disk;
external hard disk;
removable disk;
magneto-optical disk;
CD-ROM disk; or
DVD-ROM disk.

45. The method of claim 39, wherein said subject data includes any one or more of the following:
radiation therapy treatment plans;
molecular imaging planning image sets;
dose maps;
structure sets;
delivery times of said radiation dose; or
blood cell sub type distribution;
pre-treatment rate of regeneration;
pre-treatment rate of redistribution; or
subject age.

46. The method of claim 45, wherein said molecular imaging includes one of the following: computed tomography (CT), positron emission tomography (PET), ultrasound (US), magnetic resonance imaging (MRI), nuclear imaging, X-ray, single photon-emission computed tomography (SPECT), near-infrared tomography (NIRT), optical imaging, and optical computed tomography (OCT)

47. The method of claim 39, wherein said simulation model is controlled by a computer processor.

48. The method of claim 39, wherein said voxel is a three-dimensional shape within a three-dimensional matrix.

49. The method of claim 39, wherein said blood cell sub type comprises lymphocytes.

50. The method of claim 49, wherein said lymphocytes includes any one or more of the following sub types:
CD3+;
CD4+;
CD8+;
CD19+; or
CD56+.

51. The method of claim 39, wherein said at least one absorbed dose value is determined by a total blood volume, a heart-to-heart blood circulation time, a treatment delivery time, a dose delivered to moving blood, and said blood flow model.

52. The at least one absorbed dose value of claim 51, wherein said total blood volume is one of the following:
a range of about 2 to about 7 liters;
about 5 liters; or
a range of about 4 to about 6 liters.

53. The at least one absorbed dose value of claim 51, wherein said heart-to-heart blood circulation time is one of the following:
a range of about 10 seconds to about 50 seconds;
about 30 seconds; or
a range of about 20 seconds to about 40 seconds.

54. The at least one absorbed dose value of claim 51, wherein said treatment delivery time is determined by a total delivered machine units and a dose rate of energy used.

55. The at least one absorbed dose value of claim 51, wherein said dose delivered to moving blood is determined by:
dividing a total beam time into time steps;
applying said dose to a blood matrix;
rotating said blood matrix; and
randomly permuting blood.

56. The method of claim 39, wherein said blood flow model includes organ specific cardiac outputs and blood velocities.

57. The method of claim 56, wherein said blood velocities vary from a center to at least one wall of great vessels.

58. The method of claim 39, wherein said blood flow model comprises at least one logical mask, at least one dose map, at least one structure set, and at least one blood matrix.

59. The blood flow model of claim 58, wherein said at least one logical mask is provided by said at least one structure set.

60. The blood flow model of claim 59, wherein said at least one logical mask is applied for each organ.

61. The blood flow model of claim 60, wherein said at least one logical mask calculates a cross-sectional area in the z-direction.

62. The blood flow model of claim 61, wherein said cross-sectional area in the z-direction is used to shift said blood matrix.

63. The blood flow model of claim 62, wherein said blood matrix is shifted by the number of said voxels in said cross-sectional area of said at least one organ of said subject.

64. The blood flow model of claim 63, wherein an average blood density per voxel is determined for said at least one organ of said subject using the following formula:
$$v = \frac{\frac{\frac{5 Liters}{30 seconds} * CO * 1 layer}{cvoxels} * totalvoxels}{5 Liters}$$
wherein:
c is the number of voxels in one cross sectional layer,
CO is the cardiac output of the given organ, and
v is the result, wherein v is said average blood density per voxel.

65. The blood flow model of claim 64, further comprises wherein v is multiplied by a factor gv, wherein gv is a factor that accounts for higher blood density flowing through great vessels.

66. The blood flow model of claim 65, wherein said blood matrix is rotated every one second per the average blood density per voxel.

67. The method of claim 39, wherein said at least one time dependent dose is organ specific.

68. The method of claim 39, wherein said remaining blood cell sub type count is determined by the following formula:
$$N ( t ) = {N}_{0} {\sum }_{i = 0}^{i = {N}_{0}} [ 1 - K ( {D}_{i} ) ] \text{/} {N}_{0} + R ( {N}_{0} - N ( t ) ) \cdot t \qquad ( 1 )$$
wherein:
Di is the absorbed dose values for the circulating blood/lymphocyte population;
K(D) is the kill probability function for a lymphocyte dependent on the dose (D) absorbed by the lymphocyte;
N(t), remaining blood cell sub type count, at a time t following radiation therapy is calculated by:
a time dependent net release rate of new lymphocytes to the circulating blood, defined as R(N0-N(t)); and wherein:
R(N0-N(t)) represents the combined effects of release from the lymphoid organs to blood, as well as a proliferation of the existing cells, and natural death of lymphocytes in blood.

69. The method of claim 68, wherein said time dependent net release rate of new lymphocytes to the circulating blood is configured to account for age and/or pre-treatment replenishment rates.

70. The method of claim 68, wherein said kill probability function is determined by fitting at least one cell kill model to said subject data.

71. The system of claim 70, wherein said subject data includes any one or more of the following:
blood cell sub type distribution.

72. The method of claim 70, wherein said at least one cell kill model is an exponential function using the linear-quadratic model determined by the following formula:
K(D)=1−exp(−αD−βD²).
wherein:
α and β are determined using the fixed condition that K(5Gy)=0.992; and
the value K(0.5Gy) is left free to vary.

73. The method of claim 70, wherein said at least one cell kill model is a fractionated version of the linear quadratic model determined by the following formula:
K(D)=1−exp(−nd(α+βd))
wherein:
K(D) is the kill probability function for a lymphocyte dependent on the dose (D) absorbed by the lymphocyte;
α and β are determined using the fixed condition that K(5Gy)=0.992;
the value K(0.5Gy) is left free to vary; and
d is one fraction.

74. The method of claim 70, wherein said at least one cell kill model is a point to point spline fit between each data point n=0 to 5, pₙ ∈ [0,0.5,2,3,4,5] determined by the following formula:
$$K ( D ) = \frac{K ( {p}_{n + 1} ) - K ( {p}_{n} )}{{p}_{n + 1} - {p}_{n}} ( D - {p}_{n} ) + K ( {p}_{n} )$$
wherein:
the data points are as follows:
K(2Gy)=0.65;
K(3Gy)=0.88;
K(4Gy)=0.97; and
K(5Gy)=0.992;
n is equal to the value of the first data point; and
a spline point for K(0.5Gy) is left free to vary.

75. The method of claim 70, wherein said subject data further comprises a measured LYA reduction wherein the absolute value of said at least one cell kill model and said measured LYA reduction is decreased.

76. The method of claim 70, wherein said kill probability function is graphically plotted against the measurement day of said subject to calculate the slope of the trend line.

77. A non-transitory, computer readable storage medium having instructions stored thereon for use in estimating the post-treatment blood cell sub type count of a subject treated via radiation therapy that, when executed by a computer processor, cause the computer processor to:
receive subject data for a simulation model;
determine at least one time dependent dose for each voxel of at least one organ of said subject within said simulation model;
create a blood flow model for said at least one organ of said subject within said simulation model;
simulate the delivery of a radiation dose to moving blood within said subject's body using said at least one time dependent dose for each voxel of said at least one organ of said subject within said simulation model and said blood flow model;
determine at least one absorbed dose value for said subject's blood cell sub type;
calculate a remaining blood cell sub type count within said simulation model; and
transmit said remaining blood cell sub type count to a secondary source.

78. The computer readable storage medium of claim 77, wherein said secondary source includes one or more of anyone of the following:
local memory;
remote memory; or
display or graphical user interface.

79. The computer readable storage medium of claim 77, wherein said computer processor comprises at least one computer.

80. The computer readable storage medium of claim 77, wherein, when executed by the computer processor, causes the computer processor to communicate with:
a server coupled to a network;
a user interface coupled to said network; and
an application coupled to said server and/or said user interface, wherein the application is configured for executing said computer processor.

81. The computer readable storage medium of claim 78, wherein said secondary source comprises a main memory and a static memory.

82. The computer readable storage medium of claim 78, wherein said secondary source comprises one or more of anyone of the following:
electrically programmable read-only memory;
electrically erasable programmable read-only memory;
flash memory drive;
magnetic disk;
internal hard disk;
external hard disk;
removable disk;
magneto-optical disk;
CD-ROM disk; or
DVD-ROM disk.

83. The computer readable storage medium of claim 77, wherein said subject data includes any one or more of the following:
radiation therapy treatment plans;
molecular imaging planning image sets;
dose maps;
structure sets;
delivery times of said radiation dose; or
blood cell sub type distribution;
pre-treatment rate of regeneration;
pre-treatment rate of redistribution; or
subject age.

84. The computer readable storage medium of claim 83, wherein said molecular imaging includes one of the following: computed tomography (CT), positron emission tomography (PET), ultrasound (US), magnetic resonance imaging (MRI), nuclear imaging, X-ray, single photon-emission computed tomography (SPECT), near-infrared tomography (NIRT), optical imaging, and optical computed tomography (OCT)

85. The computer readable storage medium of claim 77, wherein said simulation model is controlled by said computer processor.

86. The computer readable storage medium of claim 77, wherein said voxel is a three-dimensional shape within a three-dimensional matrix.

87. The computer readable storage medium of claim 77, wherein said blood cell sub type comprises lymphocytes.

88. The computer readable storage medium of claim 87, wherein said lymphocytes includes any one or more of the following sub types:
CD3+;
CD4+;
CD8+;
CD19+; or
CD56+.

89. The computer readable storage medium of claim 77, wherein said at least one absorbed dose value is determined by a total blood volume, a heart-to-heart blood circulation time, a treatment delivery time, a dose delivered to moving blood, and said blood flow model.

90. The at least one absorbed dose value of claim 89, wherein said total blood volume is one of the following:
a range of about 2 to about 7 liters;
about 5 liters; or
a range of about 4 to about 6 liters.

91. The at least one absorbed dose value of claim 89, wherein said heart-to-heart blood circulation time is
one of the following:
a range of about 10 seconds to about 50 seconds;
about 30 seconds; or
a range of about 20 seconds to about 40 seconds.

92. The at least one absorbed dose value of claim 89, wherein said treatment delivery time is determined by a total delivered machine units and a dose rate of energy used.

93. The at least one absorbed dose value of claim 89, wherein said dose delivered to moving blood is determined by:
dividing a total beam time into time steps;
applying said dose to a blood matrix;
rotating said blood matrix; and
randomly permuting blood.

94. The computer readable storage medium of claim 77, wherein said blood flow model includes organ specific cardiac outputs and blood velocities.

95. The computer readable storage medium of claim 94, wherein said blood velocities vary from a center to at least one wall of great vessels.

96. The computer readable storage medium of claim 77, wherein said blood flow model comprises at least one logical mask, at least one dose map, at least one structure set, and at least one blood matrix.

97. The blood flow model of claim 96, wherein said at least one logical mask is provided by said at least one structure set.

98. The blood flow model of claim 97, wherein said at least one logical mask is applied for each organ.

99. The blood flow model of claim 98, wherein said at least one logical mask calculates a cross-sectional area in the z-direction.

100. The blood flow model of claim 99, wherein said cross-sectional area in the z-direction is used to shift said blood matrix.

101. The blood flow model of claim 100, wherein said blood matrix is shifted by the number of said voxels in said cross-sectional area of said at least one organ of said subject.

102. The blood flow model of claim 101, wherein an average blood density per voxel is determined for said at least one organ of said subject using the following formula:
$$v = \frac{\frac{\frac{5 Liters}{30 seconds} * CO * 1 layer}{cvoxels} * totalvoxels}{5 Liters}$$
wherein:
c is the number of voxels in one cross sectional layer,
CO is the cardiac output of the given organ, and
v is the result, wherein v is said average blood density per voxel.

103. The blood flow model of claim 102, further comprises wherein v is multiplied by a factor gv, wherein gv is a factor that accounts for higher blood density flowing through great vessels.

104. The blood flow model of claim 103, wherein said blood matrix is rotated every one second per the average blood density per voxel.

105. The computer readable storage medium of claim 77, wherein said at least one time dependent dose is organ specific.

106. The computer readable storage medium of claim 77, wherein said remaining blood cell sub type count is determined by the following formula:
$$N ( t ) = {N}_{0} {\sum }_{i = 0}^{i = {N}_{0}} [ 1 - K ( {D}_{i} ) ] \text{/} {N}_{0} + R ( {N}_{0} - N ( t ) ) \cdot t \qquad ( 1 )$$
wherein:
Di is the absorbed dose values for the circulating blood/lymphocyte population;
K(D) is the kill probability function for a lymphocyte dependent on the dose (D) absorbed by the lymphocyte;
N(t), remaining blood cell sub type count, at a time t following radiation therapy is calculated by:
a time dependent net release rate of new lymphocytes to the circulating blood, defined as R(N0-N(t)); and wherein:
R(N0-N(t)) represents the combined effects of release from the lymphoid organs to blood, as well as a proliferation of the existing cells, and natural death of lymphocytes in blood.

107. The computer readable storage medium of claim 106, wherein said time dependent net release rate of new lymphocytes to the circulating blood is configured to account for age and/or pre-treatment replenishment rates.

108. The computer readable storage medium of claim 106, wherein said kill probability function is determined by fitting at least one cell kill model to said subject data.

109. The computer readable storage medium of claim 108, wherein said subject data includes any one or more of the following:
blood cell sub type distribution.

110. The computer readable storage medium of claim 108, wherein said at least one cell kill model is an exponential function using the linear-quadratic model determined by the following formula:
K(D)=1−exp(−αD−βD²).
wherein:
α and β are determined using the fixed condition that K(5Gy)=0.992; and
the value K(0.5Gy) is left free to vary.

111. The computer readable storage medium of claim 108, wherein said at least one cell kill model is a fractionated version of the linear quadratic model determined by the following formula:
K(D)=1−exp(−nd(α+βd))
wherein:
K(D) is the kill probability function for a lymphocyte dependent on the dose (D) absorbed by the lymphocyte;
α and β are determined using the fixed condition that K(5Gy)=0.992;
the value K(0.5Gy) is left free to vary; and
d is one fraction.

112. The computer readable storage medium of claim 108, wherein said at least one cell kill model is a point to point spline fit between each data point n=0 to 5, pₙ ∈ [0,0.5,2,3,4,5] determined by the following formula:
$$K ( D ) = \frac{K ( {p}_{n + 1} ) - K ( {p}_{n} )}{{p}_{n + 1} - {p}_{n}} ( D - {p}_{n} ) + K ( {p}_{n} )$$
wherein:
the data points are as follows:
K(2Gy)=0.65;
K(3Gy)=0.88;
K(4Gy)=0.97; and
K(5Gy)=0.992;
n is equal to the value of the first data point; and
a spline point for K(0.5Gy) is left free to vary.

113. The computer readable storage medium of claim 108, wherein said subject data further comprises a measured LYA reduction wherein the absolute value of said at least one cell kill model and said measured LYA reduction is decreased.

114. The computer readable storage medium of claim 108, wherein said kill probability function is graphically plotted against the measurement day of said subject to calculate the slope of the trend line.
